(19) United States
(12) Patent Application Publication
(10) Pub. No.: US 2008/0027022 A1
(43) Pub. Date: Jan. 31, 2008

(54) METHOD TO TREAT GASTRIC LESIONS

(76) Inventors: Joel M. Linden, Charlottesville, VA (US); Masaru Odashima, Akita (JP); Jayson M. Rieger, Charlottesville, VA (US)

Correspondence Address: SCHWEGMAN, LUNDBERG & WOESSNER, P.A., P.O. BOX 2938, MINNEAPOLIS, MN, 55402, US

(21) Appl. No.: 11/672,868

(22) Filed: Feb. 8, 2007

Related U.S. Application Data
(60) Provisional application No. 60/771,267, filed on Feb. 8, 2006.

(51) Int. Cl. A61K 31/7076; A61K 45/00; A61P 1/04

(52) U.S. Cl. 514/46; 514/789

(57) ABSTRACT

The present invention provides a therapeutic method for treating gastric lesions, including administration to a patient in need thereof of an effective amount of an A_{2A} adenosine receptor agonist. The A_{2A} adenosine receptor agonist can be a compound of formula (I) as disclosed herein. The invention further provides a therapeutic method for treating the patient with an A_{2A} adenosine receptor agonist, optionally, in combination with a Type IV phosphodiesterase (PDE) inhibitor. In one embodiment, the gastric lesions are caused by, or aggravated by, the use of NSAIDS such as, for example, aspirin.

### RELATED APPLICATION

This application claims priority from U.S. Provisional Application Ser. No. 60/771,267 filed Feb. 8, 2006, the contents of the provisional application is incorporated herein by reference in its entirety.

### BACKGROUND OF THE INVENTION

Nonsteroidal anti-inflammatory drugs (NSAIDs) such as aspirin are widely used as anti-inflammatory, analgesic agents. However, gastrointestinal injury is a serious adverse effect of NSAIDs, such as aspirin, and effective strategies to protect the gastrointestinal mucosa are required. NSAIDs are believed to cause gastric lesions by inhibiting cyclooxigenase (COX), and reducing prostaglandin (PG) production. Several investigators have reported that intraperitoneal injection of anti-neutrophil serum or immunoneutralization of adhesion molecules on neutrophils and endothelial cells significantly attenuate the gastric mucosal injury induced by NSAIDs. Therefore, activation and infiltration of neutrophils into the stomach appears to contribute to gastric mucosal lesions induced by NSAIDs.

Adenosine is a primordial signaling molecule that elicits numerous physiological responses in all mammalian tissues. The receptor-mediated effects of adenosine are mediated by four G protein-coupled receptors (A₁, A_{2A}, A_{2B}, and A₃). They are variably expressed on immune cells depending on cell type and species. A_{2A} receptors are found on bone marrow derived cells including neutrophils, monocytes, macrophages, lymphocytes, platelets, and mast cells. Activation of A_{2A} receptors on immune cells produces a series of responses that, in general, can be categorized as anti-inflammatory effects. In vivo studies, have reported that activation of A_{2A} receptors attenuates ischemia/reperfusion injury in heart, lung, liver, and kidney by reducing neutrophil accumulation, superoxide generation, inhibition of endothelial adherence, and expression of the adhesion molecules. Furthermore, activation of A_{2A} receptors on human monocytes and mouse macrophages inhibits the secretion of the pro-inflammatory cytokines, IL-12 and TNF-α.

There is a need for compounds and methods for treating and preventing gastric mucosal lesions, particularly those caused by or aggravated from the use of NSAIDs.

### SUMMARY OF THE INVENTION

One embodiment provides a therapeutic method for treating gastric lesions (e.g., ulcers) induced by a variety of insults, uncluding, but not limited to, those caused by stress, bacterial infection, smoking, and/or those caused by a chemical or exogenous agent (e.g., alcohol or NSAIDs) comprising administration, to a patient in need thereof, an effective amount of an A_{2A} adenosine receptor agonist. Another embodiment provides a therapeutic method for reducing gastric mucosal lesions comprising administration, to a patient in need thereof, an effective amount of an A_{2A} adenosine receptor agonist. Another embodiment comprises treating the patient with an A_{2A} adenosine receptor agonist, optionally, in combination with a Type IV phosphodiesterase (PDE) inhibitor. In one embodiment, the gastric lesions are caused by or aggravated by the use of NSAIDS (as used herein, the term “NSAIDS” includes, but is not limited to, salycylic acids (such as Aspirin (acetylsalicylic acid), choline magnesium trisalicylate, diflunisal and/or salsalate), propionic acids (such as fenoprofen, flurbiprofen, ibuprofen, ketoprofen, naproxen and or oxaprozin), acetic acids (such as diclofenac, indomethacin, sulindac and/or tolmetin), enolic acids (such as meloxicam and/or piroxicam), fenamic acids (such as meclofenamate and/or mefenamic acid), napthylalkanones (such as nabumetone), pyranocarboxylic acids (such as etodalac), pyrroles (such as ketorolac) and/or COX-2 inhibitors (such as celecoxib, valdecoxib and/or rofecoxib).

The agonists of A_{2A} adenosine receptors of the invention can inhibit neutrophil, macrophage and T cell activation and thereby reduce inflammation caused autoimmune responses. For example, agonists of A_{2A} adenosine receptors of the invention, such as ATL146e, inhibits TNF-α and IL-1β production, neutrophil accumulation in gastric injury induced by NSAIDS (such as aspirin) without affecting mucosal prostaglandin E2 (PGE2) concentration. The effects of adenosine A_{2A} agonists can be enhanced by type IV phosphodiesterase inhibitors, such as rolipram.

One embodiment also provides compounds of the invention for use in medical therapy (e.g., for use as an adjunct in the treatment of an inflammatory response caused by gastric lesions), including gastric lesions caused by administration of NSAIDS, with A_{2A} adenosine receptor agonists, as well as the use of a compound of the invention for the manufacture of a medicament for treating gastric mucosal lesions, such as reducing inflammation caused by gastric mucosal lesions (including those caused or aggravated by NSAID use).

Another embodiment provides a method to treat an inflammatory response wherein the gastric mucosal lesions are caused by NSAIDS such as, for example, aspirin, including administering to a mammal in need of said therapy, an effective anti-inflammatory amount of an agonist of A_{2A} adenosine receptor, optionally with a PDE-IV inhibitor, such as, rolipram.

The invention also includes the use of a combination of compounds having A_{2A} adenosine receptor agonist activity with type IV phosphodiesterase inhibitors to cause synergistic decreases in the inflammatory response mediated by leukocytes.

The invention also provides a pharmaceutical composition comprising an effective amount of the compound of the invention, e.g., formula I, or a pharmaceutically acceptable salt thereof, in combination with a pharmaceutically acceptable diluent or carrier, and optionally, in combination with a Type IV phosphodiesterase (PDE) inhibitor. In one embodiment, the composition is presented as a unit dosage form.

Additionally, one embodiment provides a therapeutic method for preventing or treating a pathological condition or symptom in a mammal, such as a human, wherein the activity of A_{2A} adenosine receptors is implicated and agonism of said receptors is desired, comprising administering to a mammal in need of such therapy, an effective amount of a compound of the invention, e.g., formula I, or a pharmaceutically acceptable salt thereof. It is believed that activation of A_{2A} adenosine receptors inhibits inflammation by affecting neutrophils, mast cells, monocytes/macrophages, platelets T-cells and/or eosinophils. Inhibition of these inflammatory cells results in tissue protection following tissue insults.

### BRIEF DESCRIPTION OF THE FIGURES

FIG. 1 illustrates the Ulcer Index of rats of rats 6-hours after aspirin administration with or without pretreatment with ATL-146e (2.5 or 5 μg/kg, i.p.) (n=6 in each group). *P<0.01, **P<0.001, significantly different compared with vehicle-treated group.

FIG. 2 illustrates the effect of ATL-146e on myeloperoxidase (MPO) concentration in the gastric mucosa (n=6 in each group). *P<0.01, significantly different compared with vehicle-treated group.

FIG. 3A illustrates the effect of ATL-146e on the increase in tumor necrosis factor (TNF)-α concentration in the gastric mucosa induced by aspirin administration (n=6 in each group). *P<0.05, significantly different compared with vehicle-treated group.

FIG. 3B illustrates the effect of ATL-146e on the increase in interleukin (IL)-1β concentration in the gastric mucosa induced by aspirin administration (n=6 in each group). *P<0.01, **P<0.001, significantly different compared with vehicle-treated group.

FIG. 4 illustrates the effect of 5 μg/kg ATL-146e on gastric acid secretion. Rats were treated with or without 5 μg/kg of ATL-146e and sacrificed three hours later (n=5 in each group). *P<0.05, significantly different compared with vehicle-treated group.

FIG. 5 illustrates gastric mucosal concentrations of prostaglandin E2 in untreated rats (vehicle group), in rats treated with 200 mg/kg aspirin alone or in rats pretreated with 5 μg/kg ATL-146e (n=6 in each group). *P<0.01, significantly different compared with vehicle-treated group.

### DETAILED DESCRIPTION OF THE INVENTION

The following definitions are used, unless otherwise described. Halo is fluoro, chloro, bromo, or iodo. Alkyl, alkoxy, aralkyl, alkylaryl, etc. denote both straight and branched alkyl groups; but reference to an individual radical such as “propyl” embraces only the straight chain radical, a branched chain isomer such as “isopropyl” being specifically referred to. Aryl includes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Specific values listed below for radicals, substituents, and ranges, are for illustration only; they do not exclude other defined values or other values within defined ranges for the radicals and substituents.

Specifically, (C₁-C₈)alkyl can be methyl, ethyl, propyl, isopropyl, butyl, iso-butyl, sec-butyl, t-butyl, pentyl, 3-pentyl, hexyl, heptyl or octyl. As used herein, the term “cycloalkyl” encompasses bicycloalkyl(norbornyl, 2.2.2-bicyclooctyl, etc.) and tricycloalkyl(adamantyl, etc.), optionally comprising 1-2 N, O or S. Cycloalkyl also encompasses (cycloalkyl)alkyl. Thus, (C₃-C₆)cycloalkyl can be cyclopropyl, cyclobutyl, cyclopentyl, cyclohexyl and the like. (C₁-C₈)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl(CO₂R²) can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio, (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, puridyl (or its N-oxide), thienyl, pyrimidinyl (or its N-oxide), indolyl, isoquinolyl (or its N-oxide) or quinolyl (or its N-oxide).

Aryl denotes a phenyl radical or an ortho-fused bicyclic carbocyclic radical having about nine to ten ring atoms in which at least one ring is aromatic. Heteroaryl denotes a radical of a monocyclic aromatic ring containing five or six ring atoms consisting of carbon and 1, 2, 3, or 4 heteroatoms each selected from the group consisting of non-peroxide oxygen, sulfur, and N(Y) wherein Y is absent or is H, O, (C₁-C₈)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The term “heterocycle” generally represents a non aromatic heterocyclic group, having from 3 to about 10 ring atoms, which can be saturated or partially unsaturated, containing at least one heteroatom (e.g., 1, 2, or 3) selected from the group consisting of oxygen, nitrogen, and sulfur. Specific, “heterocycle” groups include monocyclic, bicyclic, or tricyclic groups containing one or more heteroatoms selected from the group consisting of oxygen, nitrogen, and sulfur. A “heterocycle” group also can include one or more oxo groups (═O) attached to a ring atom. Non-limiting examples of heterocycle groups include 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuelidine, thiomorpholine, and the like.

The term “alkylene” refers to a divalent straight or branched hydrocarbon chain (e.g. ethylene —CH₂CH₂—).

The term “aryl(C₁-C₈)alkylene” for example includes benzyl, phenethyl, 3-phenylpropyl, naphthylmethyl and the like.

As used herein the term “in conjunction with” refers to co-administration of an anti-rejection agent with the A_{2A} adenosine receptor agonist. The co-administration of an agent and an A_{2A} adenosine receptor agonists includes administration of the agent and agonist either simultaneously, as a mixture, or sequentially. The sequential administration of the A_{2A} adenosine receptor agonists can be prior to administration of the agent, within minutes or up to about 48 hours either before the administration of the agent. The A_{2A} adenosine receptor agonists can also be administered after the agent. In one embodiment the administration of the A_{2A} adenosine receptor agonists will be within about 24 hours such as within about 12 hours.

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, (C₁-C₈)alkyl refers to alkyl of one to eight carbon atoms, inclusive.

The compounds of the present invention are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours and “rt” for room temperature).

In one embodiment, A_{2A} agonist refers to an agent that activates the Adenosine A_{2A} receptor with a Ki of <1 μM. An A_{2A} agonist may be selective for A_{2A} (e.g., at least 10, 50, or 100/1 over another adenosine receptor subtype/A_{2A} receptor). An A_{2A} agonist may also be cross reactive with other adenosine receptor subtypes (e.g., A₁, A_{2B}, and A₃). The A_{2A} agonist may activate other receptors with a greater or lesser affinity than the A_{2A} receptor.

In one embodiment, the A_{2A} agonist of the present invention can be combined with one or more agents or additional therapeutic methods, including hydroxyurea, Decitibine, ICA 17043, transfusion, and analgesics.

In another embodiment, agonists of A_{2A} adenosine receptors that are useful in the practice of the present invention include compounds having the formula (I):

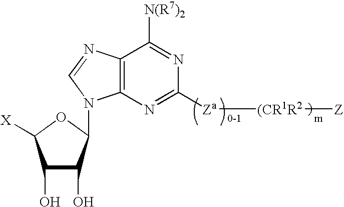

wherein

Z^{a} is C≡C, O, NH, or NHN═CR^{3a};

Z is CR³R⁴R⁵ or NR⁴R⁵; each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, or —N═NR^{b};

each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;

alternatively, R¹ and R² and the atom to which they are attached is C═O, C═S or C═NR^{d},

R⁴ and R⁵ are independently H or (C₁-C₈)alkyl;

alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from non-peroxide oxy (—O—), thio (—S—), sulfinyl (—SO—), sulfonyl (—S(O)₂—) or amine (—NR^{b}—) in the ring;

wherein R⁴ and R⁵ are independently substituted with 0-3 R⁶ groups or any ring comprising R⁴ and R⁵ is substituted with from 0 to 14 R⁶ groups; wherein each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring comprising from 1-6 carbon atoms and 1, 2, 3, or 4 heteroatoms selected from non-peroxide oxy (—O—), thio (—S—), sulfinyl (—SO—), sulfonyl (—S(O)₂—) or amine (—NR^{b}—) in the ring;

R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;

R^{3a} is hydrogen, (C₁-C₈)alkyl, or aryl;

each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});

alternatively, X is an aromatic ring of the formula:

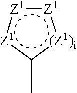

each Z¹ is non-peroxide oxy (—O—), S(O)₀₋₂, —C(R⁸)—, or amine (—NR⁸—), provided that at least one Z¹ is non-peroxide oxy (—O—), thio (—S—), sulfinyl (—SO—), sulfonyl (—S(O)₂—) or amine (—NR⁸—);

each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;

wherein any of the alkyl, cycloalkyl, heterocycle, aryl, or heteroaryl, groups of R¹, R², R³, R^{3a}, R⁶, R⁷ and R⁸ is optionally substituted on carbon with one or more (e.g. 1, 2, 3, or 4) substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)ₚ—, R^{b}R^{c}NS(O)_{P}—, and N═NR^{b};

wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;

each R^{a}, R^{b} and R^{c} is independently hydrogen, (C₁-C₁₂)alkyl, (C₁-C₈)alkoxy-(C₁-C₁₂)alkyl, (C₃-C₈)cycloalkyl, (C₁-C₈)alkylthio, amino acid, aryl, aryl(C₁-C₈)alkylene, heteroaryl, or heteroaryl(C₁-C₈)alkylene;

alternatively R^{b} and R^{c}, together with the nitrogen to which they are attached, form a pyrrolidino, piperidino, morpholino, or thiomorpholino ring;

R^{d} is hydrogen or (C₁-C₆)alkyl;

i is 1 or 2

m is 0 to 8; and

p is 0 to 2;

provided that m is at least 1 when Z is NR⁴R⁵; or

a pharmaceutically acceptable salt thereof.

Specific values listed below for radicals, substituents, and ranges, are for illustration only; they do not exclude other defined values or other values within defined ranges for the radicals and substituents.

For example, specific values include compounds having the formula (Ia):

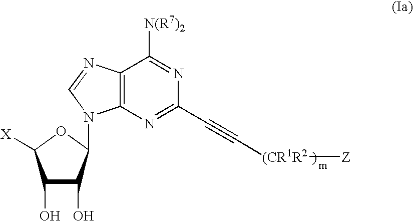

wherein

R¹ is hydrogen, —OH, —CH₂OH, —OMe, —OAc, —NH₂, —NHMe, —NMe₂ or —NHAc;

R² is hydrogen, (C₁-C₈)alkyl, cyclopropyl, cyclohexyl or benzyl;

R³ is hydrogen, OH, OMe, OAc, NH₂, NHMe, NMe₂ or NHAc;

CR⁴R⁵ or NR⁴R⁵ is optionally substituted with 0-2 R⁶ groups and is cyclopentane, cyclohexane, piperidine, dihydro-pyridine, tetrahydro-pyridine, pyridine, piperazine, tetrahydro-pyrazine, dihydro-pyrazine, pyrazine, dihydro-pyrimidine, tetrahydro-pyrimidine, hexahydro-pyrimidine, pyrazine, imidazole, dihydro-imidazole, imidazolidine, pyrazole, dihydro-pyrazole, and. pyrazolidine;

alternatively, the ring CR⁴R⁵ or NR⁴R⁵ is optionally substituted with 0-4 (e.g., 0 to 2) R⁶ groups and is selected from the group consisting of:

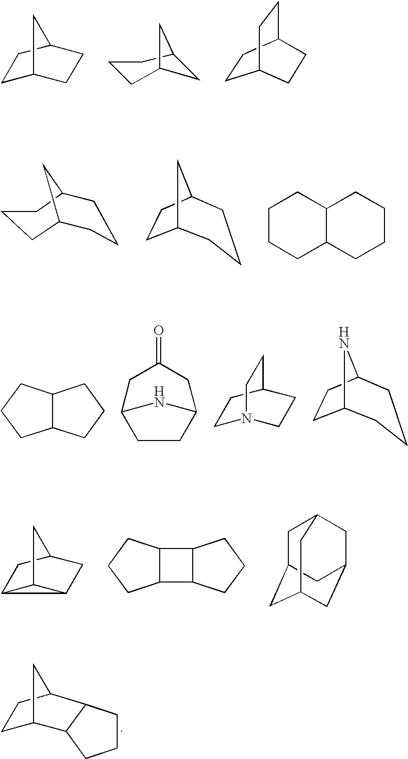

R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a}, R^{b} and R^{c}C are independently hydrogen, (C₃-C₄)-cycloalkyl, (C₁-C₈)alkyl, aryl or aryl(C₁-C₈)alkylene;

each R⁷ is independently hydrogen, alkyl (e.g,. C₁-C₈alkyl), aryl, aryl(C₁-C₈)alkylene or heteroaryl(C₁-C₈)alkylene;

R⁸ is methy, ethyl, propyl, 2-propenyl, cyclopropyl, cyclobutyl, cyclopropylmethyl, —(CH₂)₂CO₂CH₃, or —(CH₂)₂₋₃OH;

X is —CH₂OR^{a}, —CO₂R^{a}, —CH₂OC(O)R^{a}, or —C(O)NR^{b}R^{c};

alternatively X is selected from:

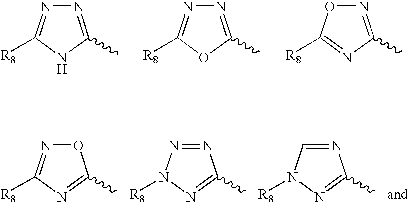

m is 0, 1 or 2;

or a pharmaceutically acceptable salt thereof.

Additional specific values include compounds having the formula (Ia), wherein:

R¹ is hydrogen, OH, OMe, or NH₂;

R² is hydrogen, methyl, ethyl or propyl;

R³ is hydrogen, OH, OMe, or NH₂;

the ring CR⁴R⁵ or NR⁴R⁵ is selected from the group consisting of:

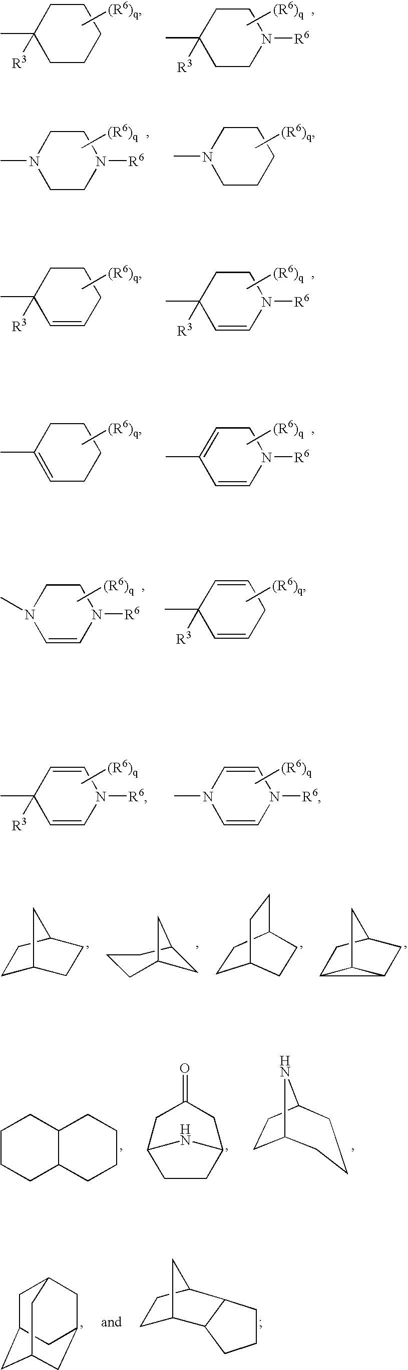

where q is from 0 to 4 (e.g., 0-2);

R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a} and R^{b} are independently hydrogen, methyl, ethyl, propyl, butyl, ethylhexyl, cyclopropyl, cyclobutyl, phenyl or benzyl;

N(R⁷)₂ is amino, methylamino, dimethylamino; ethylamino; pentylamino, diphenylethylamino, (pyridinylmethyl)amino, (pyridinyl)(methyl)amino, diethylamino or benzylamino; and,

R⁸ is methyl, ethyl, propyl, or cyclopropyl;

X is —CH₂OR^{a} or —C(O)NR^{b}R^{c};

alternatively, X is selected from:

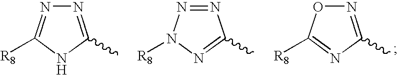

or a pharmaceutically acceptable salt thereof.

Additional specific values include compounds having the formula (Ia), wherein:

R¹ is hydrogen, OH, or NH₂;

R² is hydrogen or methyl;

R³ is hydrogen, OH, or NH₂;

the ring CR⁴R⁵ or NR⁴R⁵ is selected from the group consisting of:

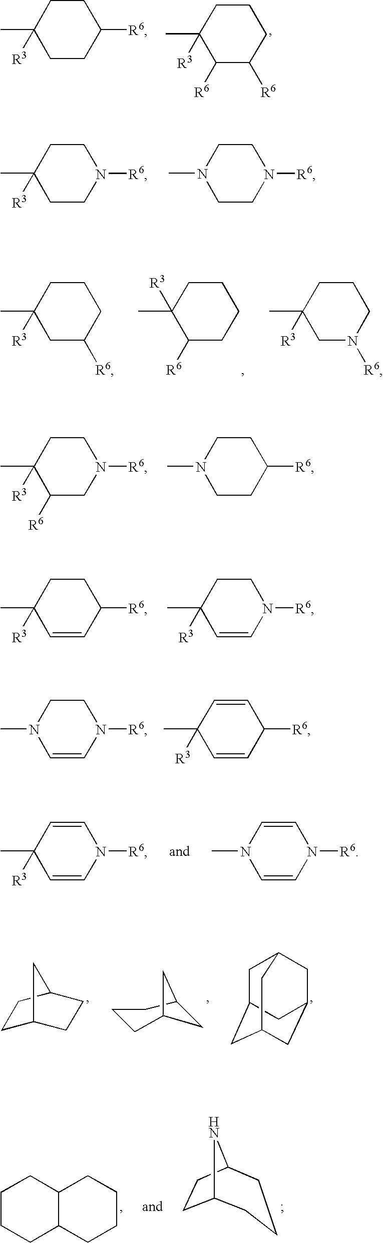

where q is from 0 to 2;

R⁶ is hydrogen, methyl, ethyl, t-butyl, , phenyl, —CO₂R^{a}—CONR^{b}R^{c}, or R^{a}C(═O)—;

R^{b} is H;

R^{a} is methyl, ethyl, propyl, butyl, pentyl, ethylhexyl cyclopropyl, and cyclobutyl;

—N(R⁷)₂ is amino, methylamino, dimethylamino; ethylamino; diethylamino or benzylamino;

or a pharmaceutically acceptable salt thereof.

Additional specific values include compounds having the formula (Ia), wherein:

R¹ is hydrogen or OH;

R² is hydrogen;

R³ is hydrogen or OH;

the ring CR⁴R⁵ or NR⁴R⁵ is selected from the group consisting of:

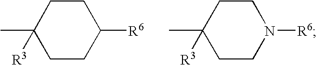

R⁶ is hydrogen, methyl, ethyl, —CO₂R^{a}, and —CONR^{b}R^{c};

R^{b} is H;

R^{a} is methyl, ethyl, i-propyl, i-butyl, tert-butyl, and cyclopropyl;

N(R⁷)₂ is amino, or methylamino;

X is —CH₂OH, 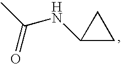
C(O)NHCH₃, or —C(O)NHCH₂CH₃;

or a pharmaceutically acceptable salt thereof.

Additional specific values include compounds wherein: the ring comprising R⁴, R⁵ and the atom to which they are connected is 2-methyl cyclohexane, 2,2-dimethylcyclohexane, 2-phenylcyclohexane, 2-ethylcyclohexane, 2,2-diethylcyclohexane, 2-tert-butyl cyclohexane, 3-methyl cyclohexane, 3,3-dimethylcyclohexane, 4-methyl cyclohexane, 4-ethylcyclohexane, 4-phenyl cyclohexane, 4-tert-butyl cyclohexane, 4-carboxymethyl cyclohexane, 4-carboxyethyl cyclohexane, 3,3,5,5-tetramethyl cyclohexane, 2,4-dimethyl cyclopentane, 4-cyclohexanecarboxylic acid, 4-cyclohexanecarboxylic acid esters, 4-methyloxyalkanoyl-cyclohexane, 4-piperidine-1-carboxylic acid methyl ester, 4-piperidine-1-carboxylic acid tert-butyl ester 4-piperidine, 4-piperazine-1-carboxylic acid methyl ester, 4-piperidine-1-carboxylic acid tert-butylester, 1-piperidine-4-carboxylic acid methyl ester, 1-piperidine-4-carboxylic acid tert-butyl ester, tert-butylester, 1-piperidine-4-carboxylic acid methyl ester, or 1-piperidine-4-carboxylic acid tert-butyl ester, 3-piperidine-1-carboxylic acid methyl ester, 3-piperidine-1-carboxylic acid tert-butyl ester, 3-piperidine, 3-piperazine-1-carboxylic acid methyl ester, 3-piperidine-1-carboxylic acid tert-butylester, 1-piperidine-3-carboxylic acid methyl ester, or 1-piperidine-3-carboxylic acid tert-butyl ester;

or a pharmaceutically acceptable salt thereof.

Additional specific values include compounds 1-33 represented in Table A or a pharmaceutically acceptable salt thereof:

TABLE A

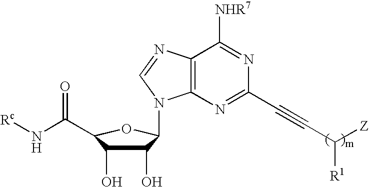

| Ex. # | R^{c} | R⁷ | —(R¹)ₘ—Z |
|---|---|---|---|
| 1. | Et | H | 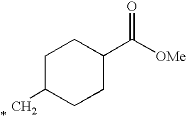 |
| 2. | cPr | H | 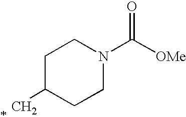 |
| 3. | Et | H | 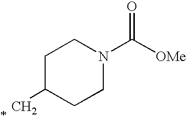 |
| 4. | Et | H | 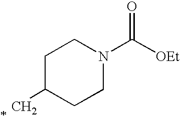 |
| 5. | Et | H | 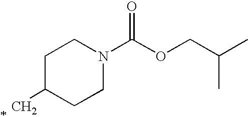 |
| 6. | Et | H | 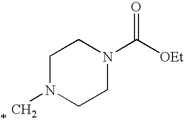 |
| 7. | Et | H | 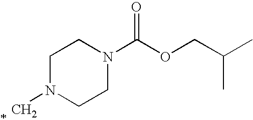 |
| 8. | Et | H | 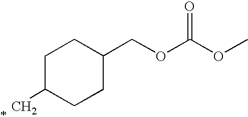 |
| 9. | Et | H | 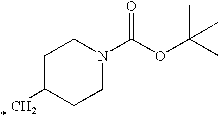 |
| 10. | Et | H | 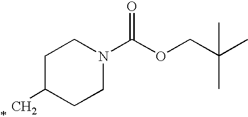 |
| 11. | cPr | H | 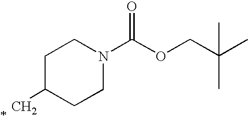 |
| 12. | Et | H | 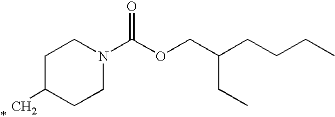 |
| 13. | cPr | H | 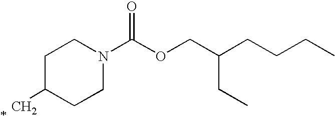 |
| 14. | Et | H | 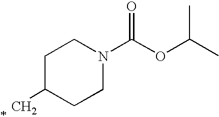 |
| 15. | cPr | H | 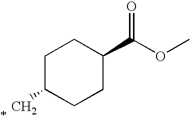 |
| 16. | Et | H | 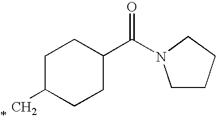 |
| 17. | Et | H | 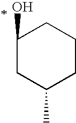 |
| 18. | Et | H | 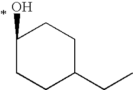 |
| 19. | Et | H | 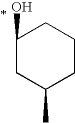 |
| 20. | Et | H | 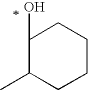 |
| 21. | Et | H | 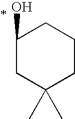 |
| 22. | cPr | H | 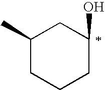 |
| 23. | Et | H | 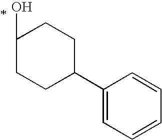 |
| 24. | Et | H | 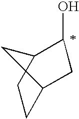 |
| 25. | cPr | H | 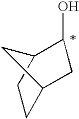 |
| 26. | cPr | H | 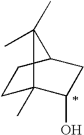 |
| 27. | Et | H | 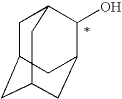 |
| 28. | cPr | H | 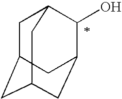 |
| 29. | Et | H | 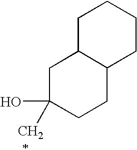 |
| 30. | cPr | H | 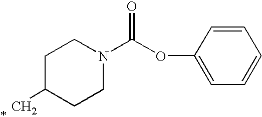 |
| 31. | Et | H | 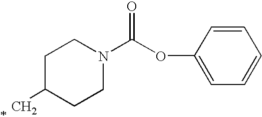 |
| 32. | cPr | H | 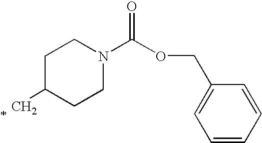 |
| 33. | Et | H | 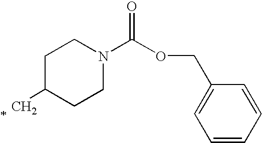 |

* signifies the point of attachment.

Additional specific values include compounds having the formula (Ib)-(Id) or a pharmaceutically acceptable salt thereof:

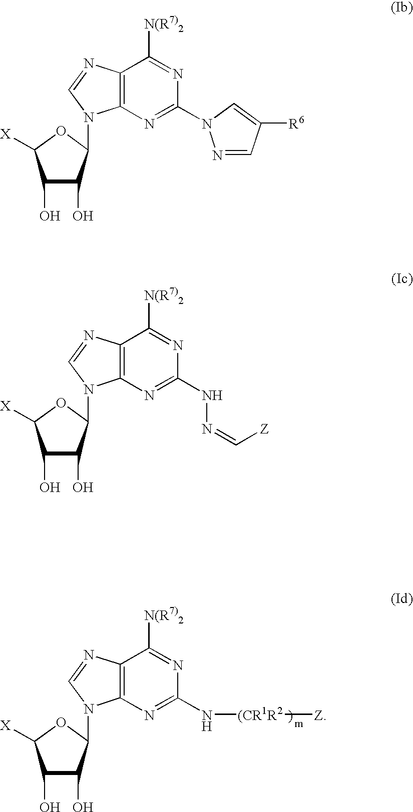

A group of specific compounds of formula (Ia) are those wherein each R⁷ is H, X is ethylaminocarbonyl, R¹ and R² are each hydrogen, and Z is a 4-piperidyl-1-carboxylic acid or ester group, wherein R^{a} is methyl, ethyl, propyl, isopropyl, isobutyl, or t-butyl, 4.

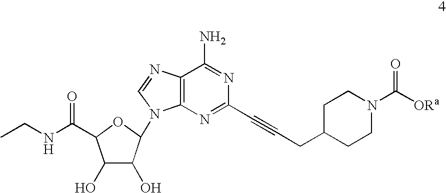

Specific A_{2A} adenosine receptor agonists suitable for use with the present invention include those described in U.S. Pat. No. 6,232,297 and in U.S. Patent Application No. 2003/0186926 A1.

Specific compounds of the invention include formula (IA)

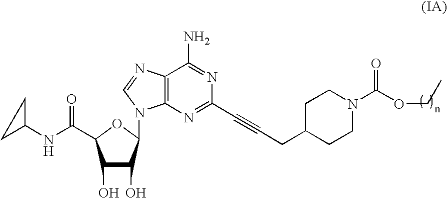

In formula (IA) n is 0, 1, 2, 3, 4, 5, 6, 7, 8, 9, 10, 11, 12, 13, 14, 15, 16, 17, or 18. In another group of specific compounds n is, 5, 6, 7, 8, 9, 10, 11, 12, 13, 14, 15, 16, 17, or 18.

Specific compounds of the invention include formula (IB)

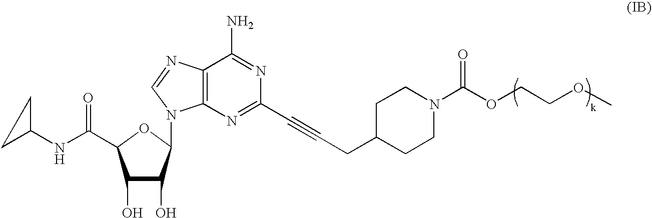

In formula (IB) k is 1, 2, 3, 4, 5, 6, 7, 8, 9, 10, 11, 12, 13, 14, 15, 16, 17, or 18.

Specific compounds of the invention include formula (IC)

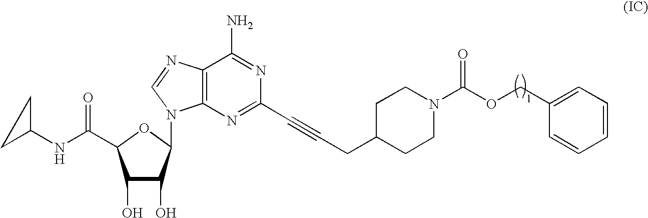

In formula (IC) 1 is 0, 1, 2, 3, or 4.

Other specific compounds of the invention include

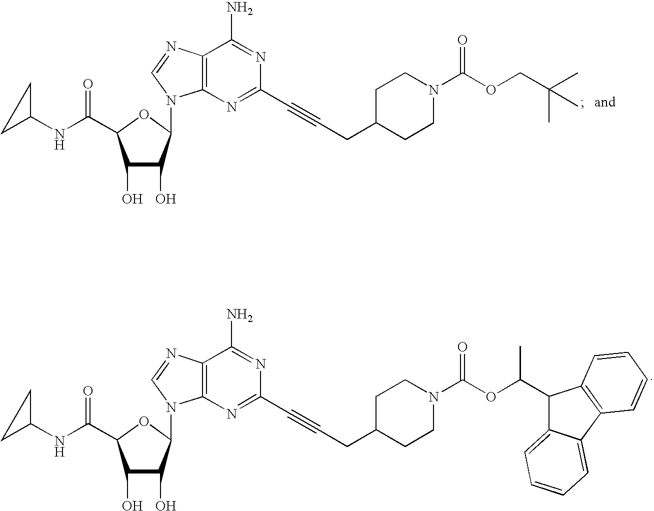

Examples of compounds useful in practicing the invention are illustrated in tables 1, 2, and 3 below:

TABLE 1

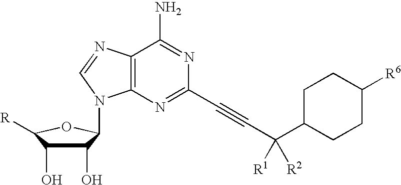

| Compound | R | R¹ | R² | R⁶ |
|---|---|---|---|---|
| ATL2037 | NECA | H | H | CH₂OH |
| MP9056 | NECA | OH | H | CH₂OH |
| ATL146a | NECA | H | H | CO₂H |
| MP9057 | NECA | OH | H | CO₂H |
| ATL146e | NECA | H | H | CO₂Me |
| MP9058 | NECA | OH | H | CO₂Me |
| JR2145 | CH₂OH | H | H | CO₂Me |
| MP9059 | CH₂OH | OH | H | CO₂Me |
| ATL193 | NECA | H | H | CH₂OAc |
| MP9060 | NECA | OH | H | CH₂OAc |
| JR2147 | CH₂OH | H | H | CH₂OAc |
| MP9061 | CH₂OH | OH | H | CH₂OAc |
| JR3023 | NECA | H | H | CH₂N(CH₃)₂ |
| MP9062 | NECA | OH | H | CH₂N(CH₃)₂ |
| JR3021 | NECA | H | H | COOCH₂CH₂NHBoc |
| MP9063 | NECA | OH | H | COOCH₂CH₂NHBoc |
| JR3033 | NECA | H | H | COOCH₂CH₂NH₂ |
| MP9064 | NECA | OH | H | COOCH₂CH₂NH₂ |
| JR3037 | NECA | H | H | CONHCH₂CH₃ |
| MP9065 | NECA | OH | H | CONHCH₂CH₃ |
| JR3055 | NECA | H | H | CONH₂ |
| MP9072 | NECA | OH | H | CONH₂ |
| JR3065 | NECA | H | H | CONHMe |
| MP9066 | NECA | OH | H | CONHMe |
| JR3067B | NECA | H | H | Me, cis CO₂Me |
| MP9067 | NECA | OH | H | Me, cis CO₂Me |
| JR3067A | NECA | H | H | Me, trans CO₂Me |
| MP9068 | NECA | OH | H | Me, trans CO₂Me |
| JR3087 | NECA | H | H | CH₂CH₃ |
| MP9069 | NECA | OH | H | CH₂CH₃ |
| JR3159A | NECA | OH | H | H |
| JR3159B | NECA | OH | H | H |
| JR3119 | NECA | H | H | COCH₃ |
| MP9070 | NECA | OH | H | COCH₃ |
| JR3121 | NECA | H | H | CHCH₃(OH) |
| MP9071 | NECA | OH | H | CHCH₃(OH) |
| JR3139 | NECA | OH | C₆H₁₁ | H |

NECA = CH₃CH₂N(H)C(O)—

TABLE 2

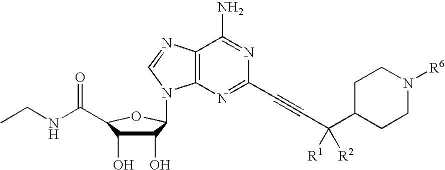

| Compound | R¹ | R² | R⁶ |
|---|---|---|---|
| JR3261 | H | H | H |
| JR3259 | H | H | CO₂tBu |
| JR3269 | H | H | CO₂Et |
| JR4011 | H | H | CO₂iBu |
| JR4009 | H | H | CO₂iPr |
| JR4007 | H | H | COMe |
| JR4051 | H | H | COC(CH₃)₃ |
| JR4047 | H | H | COCH₂(CH₃)₃ |
| MP9047 | H | H | COCH₃ |
| MP9048 | H | H | C(O)N(CH₃)₂ |
| MP9049 | H | H | C(O)N(CH₃)Et |
| MP9050 | H | H | C(O)N(CH₃)iPr |
| MP9051 | H | H | C(O)N(CH₃)iBu |
| MP9052 | H | H | C(O)NH(CH₃) |
| MP9053 | H | H | C(O)NH(Et) |
| MP9054 | H | H | C(O)NH(iPr) |
| MP9055 | H | H | C(O)NH(iBu) |
| TX3261 | OH | H | H |
| TX3259 | OH | H | CO₂tBu |
| TX3269 | OH | H | CO₂Et |
| TX4011 | OH | H | CO₂iBu |
| TX4009 | OH | H | CO₂iPr |
| TX4007 | OH | H | COMe |
| TX4051 | OH | H | COC(CH₃)₃ |
| TX4047 | OH | H | COCH₂(CH₃)₃ |
| TX9047 | OH | H | COCH₃ |
| TX9048 | OH | H | C(O)N(CH₃)₂ |
| TX9049 | OH | H | C(O)N(CH₃)Et |
| TX9050 | OH | H | C(O)N(CH₃)iPr |
| TX9051 | OH | H | C(O)N(CH₃)iBu |
| TX9052 | OH | H | C(O)NH(CH₃) |
| TX9053 | OH | H | C(O)NH(Et) |
| TX9054 | OH | H | C(O)NH(iPr) |
| TX9055 | OH | H | C(O)NH(iBu) |

TABLE 3

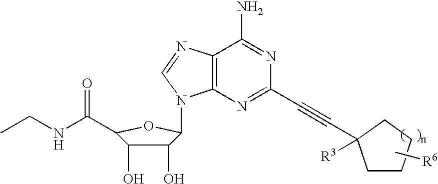

| Compound | n | R³ | R⁶ |
|---|---|---|---|
| JR3135 | 1 | OH | H |
| JR3089 | 2 | OH | H |
| JR3205 | 2 | NH₂ | H |
| JR3177A | 2 | OH | 2-CH₃ |
| JR3177B | 2 | OH | 2-CH₃ |
| JR3181A | 2 | OH | 2-CH₃ |
| JR3181B | 2 | OH | 2-CH₃ |
| JR3227 | 2 | OH | 2-C(CH₃)₃ |
| JR9876 | 2 | OH | 2-C₆H₅ |
| JR3179 | 2 | OH | 3-CH₃ |
| JR3221 | 2 | OH (R) | 3-CH₃ (R) |
| ATL 203 | 2 | OH (S) | 3-CH₃ (R) |
| MP9041 | 2 | OH (R) | 3-CH₃ (S) |
| MP9042 | 2 | OH (S) | 3-CH₃ (S) |
| JR3201B | 2 | OH | 3-(CH₃)₂ |
| MP9043 | 2 | OH (R) | 3-CH₂CH₃ (R) |
| MP9044 | 2 | OH (S) | 3-CH₂CH₃ (R) |
| MP9045 | 2 | OH (R) | 3-CH₂CH₃ (S) |
| MP9046 | 2 | OH (S) | 3-CH₂CH₃ (S) |
| JR3163 | 2 | OH | 3-(CH₃)₂, 5-(CH₃)₂ |
| JR9875 | 2 | OH | 4-CH₃ |
| JR3149 | 2 | OH | 4-C₂H₅ |
| JR3203 | 2 | OH | 4-C(CH₃)₃ |
| JR3161 | 2 | OH | 4-C₆H₅ |

In another embodiment, agonists of A_{2A} adenosine receptors that are useful in the practice of the present invention include compounds having the formula (II):

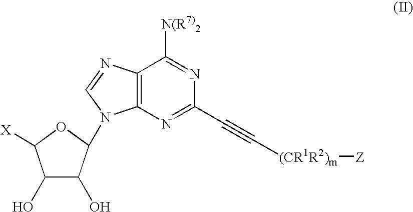

wherein Z is CR³R⁴R⁵; each R¹, R² and R³ is hydrogen; R⁴ and R⁵ together with the carbon atom to which they are attached form a cycloalkyl ring having 3, 4, 5, 6, 7, 8, 9 or 10 ring atoms; and

wherein the ring comprising R⁴ and R⁵ is substituted with —(CH₂)₀₋₆—Y; where Y is —CH₂OR^{a}, —CO₂R^{a}, —OC(O)R^{a}, —CH₂OC(O)R^{a}, —CH₂OC(O)R^{a}, —C(O)NR^{b}R^{c}, —CH₂SR^{a}, —C(S)OR^{a}, —OC(S)R^{a}, —CH₂OC(S)R^{a} or C(S)NR^{b}R^{c} or —CH₂N(R^{b})(R^{c});

each R⁷ is independently hydrogen, (C₁-C₈)alkyl, (C₃-C₈)cycloalkyl, aryl or aryl(C₁-C₈)alkylene;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, C(S)NR^{b}R^{c}C or —CH₂N(R^{b})(R^{c});

each R^{a}, R^{b} and R^{c} is independently hydrogen, (C₁-C₈)alkyl, or (C₁-C₈)alkyl substituted with 1-3 (C₁-C₈)alkoxy, (C₃-C₈)cycloalkyl, (C₁-C₈)alkylthio, amino acid, aryl, aryl(C₁-C₈)alkylene, heteroaryl, or heteroaryl(C₁-C₈)alkylene; or R^{b} and R^{c}, together with the nitrogen to which they are attached, form a pyrrolidino, piperidino, morpholino, or thiomorpholino ring; and m is 0 to about 6; or a pharmaceutically acceptable salt thereof.

A specific value for —N(R⁷)₂ is amino, monomethylamino or cyclopropylamino.

A specific value for Z is carboxy- or —(C₁-C₄)alkoxycarbonyl-cyclohexyl(C₁-C₄)alkyl.

A specific value for R^{a} is H or (C₁-C₄)alkyl, i.e., methyl or ethyl.

A specific value for R^{b} is H, methyl or phenyl.

A specific value for R^{c} is H, methyl or phenyl.

A specific value for —(CR¹R²)ₘ— is —CH₂— or —CH₂—CH₂—.

A specific value for X is CO₂R^{a}, (C₂-C₅)alkanoylmethyl or amido.

A specific value for Y is CO₂R^{a}, (C₂-C₅)alkanoylmethyl or amido.

A specific value for m is 1.

Specific compounds useful for practicing the invention are compounds JR3259, JR3269, JR4011, JR4009, JR-1085 and JR4007.

Specific A_{2A} adenosine receptor agonists suitable for use with the present invention having formula (II) include those described in U.S. Pat. No. 6,232,297. Specific compounds of formula (II) are those wherein each R⁷ is H, X is ethylaminocarbonyl and Z is 4-carboxycyclohexylmethyl (DWH-146a), Z is 4-methoxycarbonylcyclohexylmethyl (DWH-146e), Z is 4-isopropylcarbonyl-cyclohexylmethyl (AB-1), Z is 4-acetoxymethyl-cyclohexylmethyl (JMR-193) or Z is 4-pyrrolidine-1-carbonylcyclohexylmethyl (AB-3). Additional compounds useful in practicing the invention are depicted below.

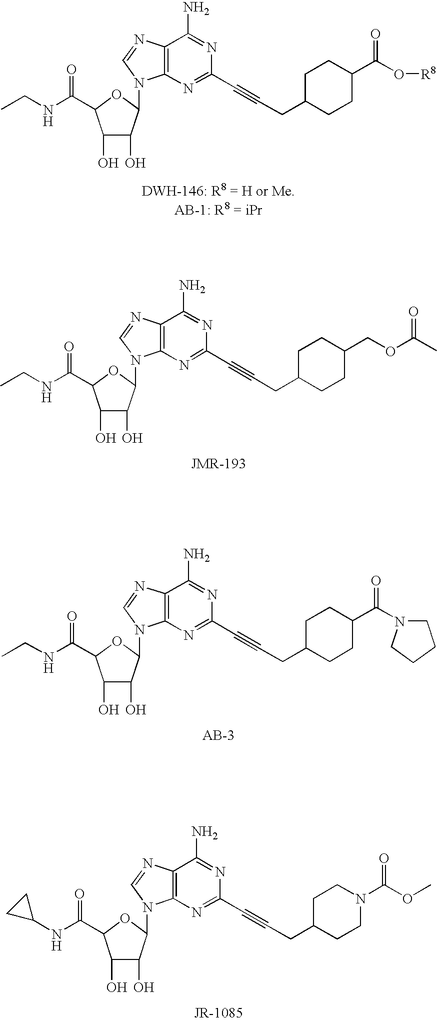

The specific A_{2A} adenosine receptor agonists suitable for use with the present invention having formula (II) include those described in U.S. Pat. No. 6,232,297. These compounds, having formula (II), can be prepared according to the methods described therein.

Another specific group of agonists of A_{2A} adenosine receptors that are useful in the practice of the present invention include compounds having the general formula (III):

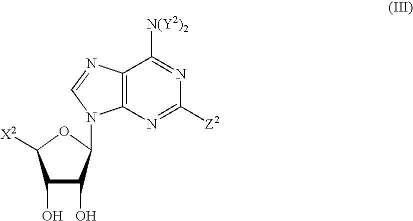

wherein Z² is a group selected from the group consisting of —OR¹², —NR¹³R¹⁴, a —C≡C—Z³, and —NH—N═R¹⁷;

each Y² is individually H, C₁-C₆ alkyl, C₃-C₇ cycloalkyl, phenyl or phenyl C₁-C₃ alkyl;

R¹² is

C₁₋₄-alkyl; C₁₋₄-alkyl substituted with one or more C₁₋₄-alkoxy groups, halogens (fluorine, chlorine or bromine), hydroxy groups, amino groups, mono(C₁₋₄-alkyl)amino groups, di(C₁₋₄-alkyl)amino groups or C₆₋₁₀-aryl groups wherein the aryl groups may be substituted with one or more halogens (fluorine, chlorine or bromine), C₁₋₄-alkyl groups, hydroxy groups, amino groups, mono(C₁₋₄-alkyl)amino groups or di(C₁₋₄-alkyl)amino groups); or

C₆₋₁₀-aryl or C₆₋₁₀-aryl substituted with one or more halogens (fluorine, chlorine or bromine), hydroxy groups, amino groups, mono(C₁₋₄-alkyl)amino groups, di(C₁₋₄-alkyl)amino groups or C₁₋₄-alkyl groups;

one of R¹³ and R¹⁴ has the same meaning as R¹² and the other is hydrogen; and

R¹⁷ is a group having the formula (i)

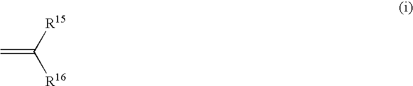

wherein each of R¹⁵ and R¹⁶ independently may be hydrogen, (C₃-C₇)cycloalkyl or any of the meanings of R¹², provided that R¹⁵ and R¹⁶ are not both hydrogen;

X² is CH₂OH, CH₃, CO₂R²⁰ or C(═O)NR²¹R²² wherein R²⁰ has the same meaning as R¹³ and wherein R²¹ and R²² have the same meanings as R¹⁵ and R¹⁶ or R²¹ and R²² are both H;

Z³ has one of the following meanings:

C₆-C₁₀ aryl, optionally substituted with one to three halogen atoms, C₁-C₆ alkyl, C₁-C₆ haloalkyl, C₁-C₆ alkoxy, C₁-C₆ haloalkoxy, C₂-C₆ alkoxycarbonyl, C₂-C₆ alkoxyalkyl, C₁-C₆ alkylthio, thio, CHO, cyanomethyl, nitro, cyano, hydroxy, carboxy, C₂-C₆ acyl, amino C₁-C₃ monoalkylamino, C₂-C₆ dialkylamino, methylenedioxy or aminocarbonyl;

a group of formula —(CH₂)_{q}-Het wherein q is 0 or an integer from 1 to 3 and Het is 5 or 6 membered heterocyclic aromatic or non-aromatic ring, optionally benzocondensed, containing 1 to 3 heteroatoms selected from non-peroxide oxygen, nitrogen or sulphur, linked through a carbon atom or through a nitrogen atom;

C₃-C₇ cycloalkyl optionally containing unsaturation or C₂-C₄ alkenyl;
wherein 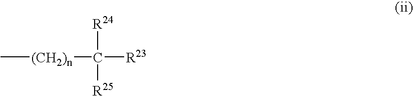
   R²³ is hydrogen, methyl or phenyl;
   R²⁴ is hydrogen, C₁-C₆ linear or branched alkyl, C₅-C₆ cycloalkyl or C₃-C₇ cycloalkenyl, phenyl-C₁-C₂-alkyl or R²³ and R²⁴, taken together, form a 5 or 6-membered carbocyclic ring or R²⁵ is hydrogen and R²³ and R²⁴, taken together, form an oxo group or a corresponding acetalic derivative;
   R²⁵ is OH, NH₂ dialkylamino, halogen, cyano; and n is 0 or 1 to 4; or
a) C₁-C₁₆ alkyl, optionally comprising 1-2 double bonds, O, S or NY²;

or a pharmaceutically acceptable salt thereof.

Specific C₆₋₁₀-aryl groups include phenyl and naphthyl.

In one embodiment, in the compound of formula (I), Z² is a group of the formula (iii)
—O—(CH₂)ₙ—Ar (iii)

wherein n is an integer from 1-4, preferably 2, and Ar is a phenyl group, tolyl group, naphthyl group, xylyl group or mesityl group. In one embodiment Ar is a para-tolyl group and n=2.

In one embodiment, in the compound of formula (I), Z² is a group of the formula (iv)
NHN═CHCy (iv)

wherein Cy is a C₃₋₇-cycloalkyl group, such as cyclohexyl or a C₁₋₄ alkyl group, including isopropyl.

In another embodiment, in the compound of formula (I), Z² is a group of the formula (vii)
C≡CZ³ (v)

wherein Z³ is C₃-C₁₆ alkyl, hydroxy C₂-C₆ alkyl or (phenyl) (hydroxymethyl).

Specific examples of such compounds of formula (I) include those shown below:

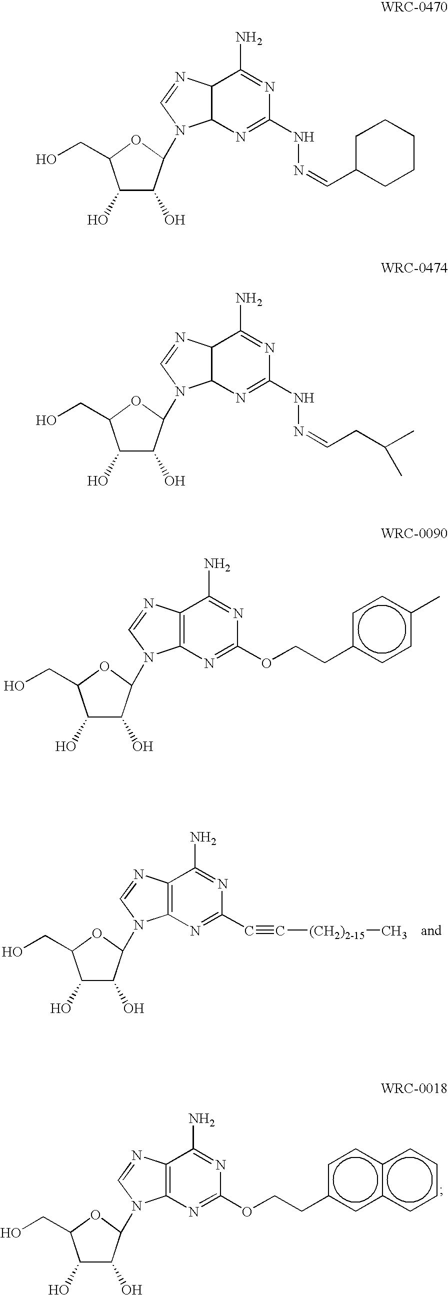

wherein the H on CH₂OH can optionally be replaced by ethylaminocarbonyl. Of these specific examples, WRC-0474[SHA 211] and WRC-0470 are particularly preferred.

Such compounds may be synthesized as described in: Olsson et al. (U.S. Pat. Nos. 5,140,015 and 5,278,150); Cristalli (U.S. Pat. No. 5,593,975); Miyasaka et al. (U.S. Pat. No. 4,956,345); Hutchinson, A. J. et al., J. Pharmacol. Exp. Ther., 251, 47 (1989); Olsson, R. A. et al., J. Med. Chem., 29, 1683 (1986); Bridges, A. J. et al., J. Med. Chem., 31, 1282 (1988); Hutchinson, A. J. et al., J. Med. Chem., 33, 1919 (1990); Ukeeda, M. et al., J. Med. Chem., 34, 1334 (1991); Francis, J. E. et al., J. Med. Chem., 34, 2570 (1991); Yoneyama, F. et al., Eur. J. Pharmacol., 213, 199-204 (1992); Peet, N. P. et al., J. Med. Chem., 35, 3263 (1992); and Cristalli, G. et al., J. Med. Chem., 35, 2363 (1992); all of which are incorporated herein by reference.

Another embodiment includes compounds having formula (III) where Z² is a group having formula (vi):

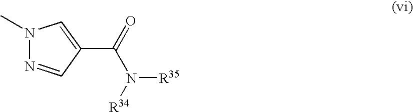

wherein R³⁴ and R³⁵ are independently H, C₁-C₆ alkyl, C₃-C₇ cycloalkyl, phenyl, phenyl C₁-C₃ alkyl or R³⁴ and R³⁵ taken together with the nitrogen atom are a 5- or 6-membered heterocyclic ring containing 1-2 heteroatoms selected from non-peroxide oxygen, nitrogen (N(R¹³)) or sulphur atoms. In one embodiment one of R³⁴ and R³⁵ is hydrogen and the other is ethyl, methyl or propyl. In another embodiment one of R³⁴ and R³⁵ is hydrogen and the other is ethyl or methyl.

The 2-(pyrazol-1-yl)adenosine compounds of the invention, wherein Z² is a group having formula (vi), can be prepared by reacting a 2-chloro- or 2-iodo adenosine derivative with an 1H-pyrazole-4-carboxamides compound having formula (vii):

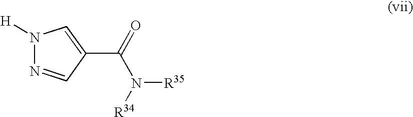

where R³⁴ and R³⁵ are as described above, wherein selective protection/deprotection of the amido group is used as needed. A specific pyrazole derivative useful in practicing this invention is a compound having the formula:

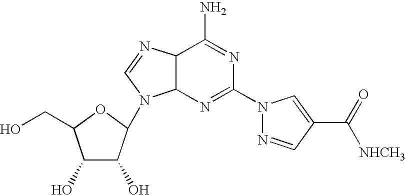

The 1H-pyrazole-4-carboxamides can be prepared starting with 1H-pyrazole-4-carboxylic acid, available from Aldrich Chemical Co. In the first step, the acid is converted to an ester, e.g., a methyl or ethyl ester. The ester is converted to the amide via aminolysis, e.g., with methylamine to form the methyl amide. The pyrazole-4-carboxamide will react with the 2-halopurines in the presence of a strong base to provide the 2-(pyrazol-1-yl)adenosine compounds having formula (III).

Another specific group of agonists of A_{2A} adenosine receptors that are useful in the practice of the present invention include compounds having the general formula (IV): 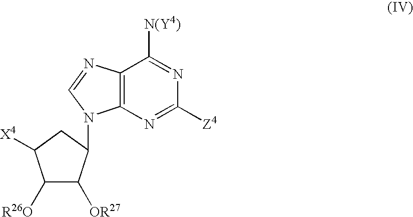
wherein Z⁴ is —NR²⁸R²⁹;

R²⁸ is hydrogen or (C₁-C₄) alkyl; and R²⁹ is
a) (C₁-C₄) alkyl;
b) (C₁-C₄)alkyl substituted with one or more (C₁-C₄)alkoxy, halogen, hydroxy, amino, mono((C₁-C₄) alkyl)amino, di((C₁-C₄)alkyl)amino or (C₆-C₁₀) aryl wherein aryl is optionally substituted with one or more halogen, hydroxy, amino, (C₁-C₄)alkyl, R³⁰OOC—((C₁-C₄)alkyl)-, R³¹R³²NC(═O)—((C₁-C₄)alkyl)-, mono((C₁-C₄)alkyl)amino or di((C₁-C₄)alkyl)amino;
c) (C₆-C₁₀)aryl; or
d) (C₆-C₁₀)aryl substituted with one or more halogen, hydroxy, amino, mono((C₁-C₄)alkyl)amino, di((C₁-C₄)alkyl)amino or (C₁-C₄)alkyl;

wherein each Y⁴ is individually H, (C₁-C₆)alkyl, (C₃-C₇)cycloalkyl, phenyl or phenyl(C₁-C₃)alkyl; and X⁴ is —C(═O)NR³¹R³², —COOR³⁰, or —CH₂OR³⁰;

wherein each of R³¹ and R³² are independently; hydrogen; C₃₋₇-cycloalkyl; (C₁-C₄)alkyl; (C₁-C₄)alkyl substituted with one or more (C₁-C₄)alkoxy, halogen, hydroxy, —COOR³³, amino, mono((C₁-C₄)alkyl)amino, di((C₁-C₄)alkyl)amino or (C₆-C₁₀)aryl wherein aryl is optionally substituted with one or more halogen, (C₁-C₄)alkyl, hydroxy, amino, mono((C₁-C₄) alkyl)amino or di((C₁-C₄) alkyl)amino; (C₆-C₁₀)aryl; or (C₆-C₁₀)aryl substituted with one or more halogen, hydroxy, amino, mono((C₁-C₄)alkyl)amino, di((C₁-C₄)alkyl)amino or (C₁-C₄)alkyl;

R²⁶ and R²⁷ independently represent hydrogen, lower alkanoyl, lower alkoxy-lower alkanoyl, aroyl, carbamoyl or mono- or di-lower alkylcarbamoyl; and R³⁰ and R³³ are independently hydrogen, (C₁-C₄)alkyl, (C₆-C₁₀)aryl or (C₆-C₁₀)aryl((C₁-C₄)alkyl); or a pharmaceutically acceptable salt thereof.

In one embodiment of formula (IV), at least one of R²⁸ and R²⁹ is (C₁-C₄)alkyl substituted with one or more (C₁-C₄)alkoxy, halogen, hydroxy, amino, mono((C₁-C₄)alkyl)amino, di((C₁-C₄)alkyl)amino or (C₆-C₁₀)aryl wherein aryl is optionally substituted with one or more halogen, hydroxy, amino, (C₁-C₄)alkyl, R³⁰OOC—(C₁-C₄)alkyl, mono((C₁-C₄)alkyl)amino or di((C₁-C₄)alkyl)amino.

In another embodiment, at least one of R³¹ and R³² is C₁₋₄-alkyl substituted with one or more (C₁-C₄)alkoxy, halogen, hydroxy, amino, mono((C₁-C₄)alkyl)amino, di((C₁-C₄)alkyl)amino or C₆₋₁₀-aryl wherein aryl is optionally substituted with one or more halogen, hydroxy, amino, (C₁-C₄)alkyl, R³⁰OOC—(C₁-C₄)alkylene-, mono((C₁-C₄)alkyl)amino or di((C₁-C₄)alkyl)amino.

In another embodiment, at least one of R²⁸ and R²⁹ is C₆₋₁₀-aryl substituted with one or more halogen, hydroxy, amino, mono((C₁-C₄)alkyl)amino, di((C₁-C₄)alkyl)amino or (C₁-C₄)alkyl.

In another embodiment, at least one of R³¹ and R³² is C₆₋₁₀-aryl substituted with one or more halogen, hydroxy, amino, mono((C₁-C₄)alkyl)amino, di((C₁-C₄)alkyl)amino or (C₁-C₄)alkyl.

In a specific combination, R³¹ is hydrogen and R³² is (C₁-C₄)alkyl, cyclopropyl or hydroxy-(C₂-C₄)alkyl. A specific R²⁸ group is (C₁-C₄)alkyl substituted with (C₆-C₁₀)aryl, that is in turn substituted with R³⁰O(O)C—(C₁-C₄)alkylene-.

A specific compound having formula (IV) is:

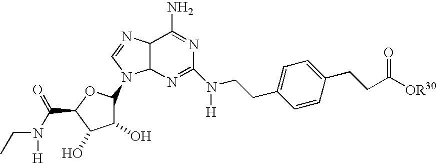

wherein R³⁰ is hydrogen, methyl, ethyl, n-propyl or isopropyl. One embodiment provides a compound wherein the R³⁰ group is methyl or ethyl. Another embodiment provides a compound wherein the R³⁰ group is methyl.

Two compounds that are particularly useful in practicing the present invention have the formula:

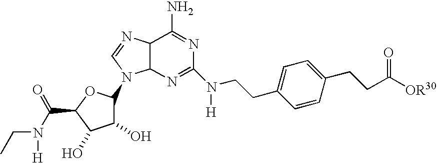

wherein R³⁰ is hydrogen (acid, CGS21680) and where R³⁰ is methyl (ester, JR2171).

The compounds of the invention having formula (IV) may be synthesized as described in: U.S. Pat. No. 4,968,697 or J. Med. Chem., 33, 1919-1924, (1990).

Another agonist compound that is useful in the present invention is IB-MECA, shown below.

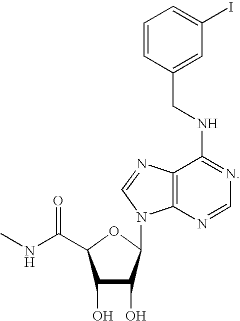

It will be appreciated by those skilled in the art that the compounds of formulas (I), (II), (III), and (IV) (as described herein) have more than one chiral center and may be isolated in optically active and racemic forms. In one embodiment, the riboside moiety of the compounds is derived from D-ribose, i.e., the 3′,4′-hydroxyl groups are alpha to the sugar ring and the 2′ and 5′ groups is beta (3R, 4S, 2R, 5S). When the two groups on the cyclohexyl group are in the 1- and 4-position, they are preferably trans. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

Specifically, the invention also provides the use of a compound of formula (I) or a pharmaceutically acceptable salt thereof to prepare a medicament for treating systemic intoxification in a mammal (e.g., a human).

Specifically, the invention also provides the use of a compound of formula (I) or a pharmaceutically acceptable salt thereof to prepare a medicament for treating inflammation caused by bacterial, fungal or viral infections and the inflammation caused by the treatment of these infections, e.g., by the death of the bacterial or viral cells in a mammal (e.g., a human).

The present method also includes the administration of a Type IV phosphodiesterase (PDE) inhibitor in combination with compounds having formulae (I), (II), (III), or (IV). The combination of the compounds of the invention with type IV phosphodiesterase inhibitor provides synergistic decreases in the inflammatory response of immune cells. Examples of Type IV phosphodiesterase (PDE) inhibitors include those disclosed in U.S. Pat. No. 4,193,926, and WO 92-079778, and Molnar-Kimber, K. L. et al., J. Immunol., 150, 295A (1993), all of which are incorporated herein by reference.

Suitable Type IV phosphodiesterase (PDE) inhibitors include racemic and optically active 4-(polyalkoxyphenyl)-2-pyrrolidones of general formula (VI):

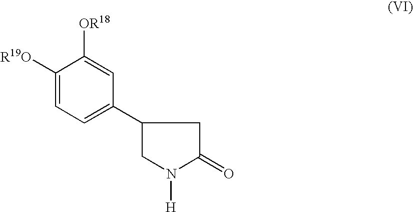

(disclosed and described in U.S. Pat. No. 4,193,926) wherein R¹⁸ and R¹⁹ are independently the same or different and are hydrocarbon radicals having up to 18 carbon atoms with at least one being other than methyl, a heterocyclic ring, or alkyl of 1-5 carbon atoms which is substituted by one or more of halogen atoms, hydroxy, carboxy, alkoxy, alkoxycarbonyl or an amino group or amino.

Examples of hydrocarbon R¹⁸ and R¹⁹ groups are saturated and unsaturated, straight-chain and branched alkyl of 1-18, such as 1-5, carbon atoms, cycloalkyl and cycloalkylalkyl, such as 3-7 carbon atoms, and aryl and aralkyl, such as of 6-10 carbon atoms, especially monocyclic.

Rolipram is an example of a suitable Type IV phosphodiesterase or PDE inhibitor included within the above formula. Rolipram has the following formula:

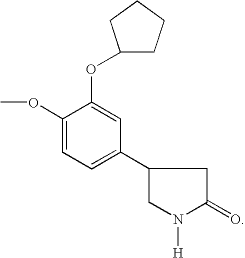

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds of the present invention can conveniently be administered in a pharmaceutical composition containing the compound in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally (for example, by intravenous, intraperitoneal or intramuscular injection), topically, orally, or rectally.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, isotonic agents, for example, sugars, buffers or sodium chloride, will be included. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation include vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The compound is conveniently administered in unit dosage form; for example, containing about 0.05 mg to about 500 mg, conveniently about 0.1 mg to about 250 mg, most conveniently, about 1 mg to about 150 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compositions can conveniently be administered orally, sublingually, transdermally, or parenterally at dose levels of about 0.01 to about 150 μg/kg, about 0.1 to about 50 μg/kg, and about 0.1 to about 10 μg/kg of mammal body weight.

For parenteral administration the compounds are presented in aqueous solution in a concentration of from about 0.1 to about 10%, such as about 0.1 to about 7%. The solution may contain other ingredients, such as emulsifiers, antioxidants or buffers.

The preparation of compounds useful in practicing the present invention are disclosed in U.S. patent application Ser. No. 10/236,379, filed Oct. 1, 2002, and can generally be prepared as illustrated in Schemes 1A and 1B below. Starting materials can be prepared by procedures described in these schemes, procedures described in the General methods below or by procedures that would be well known to one of ordinary skill in organic chemistry. The variables used in Schemes 1A and Scheme 1B are as defined herein or as in the claims.

The preparation of alkynyl cycloalkanols is illustrated in Scheme 1A. A solution of an appropriate cycloalkanone (where j is from 0-5) is prepared in a solvent such as THF. A solution of a suitable ethynylmagnesium halide compound in a solvent is added to the cycloalkanone. After addition, the solution is allowed to stir at about 20 C. for about 20 hours. The reaction is monitored via TLC until the starting material is consumed. The reaction is quenched with water, filtered over a plug of sand and silica, washed with a solvent, such as EtOAc, and evaporated to provide the product. Typically, two products are formed, the isomers formed by the axial/equatorial addition of the alkyne (where m is as defined above, and the sum of m1 and m2 is from 0 to about 7) to the ketone. The compounds are purified via flash chromatography using EtOAc/Hexanes to provide the product.

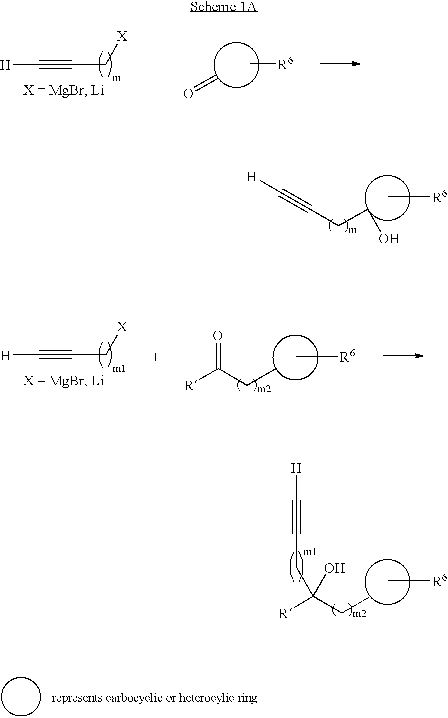

In accordance with one embodiment of the present invention a composition comprising an agonist of A_{2A} AR is administered to a patient to treat gastric lesions. As used herein the term “treating” includes prophylaxis of the specific disorder or condition, or alleviation of the symptoms associated with a specific disorder or condition and/or preventing or eliminating said symptoms.

The preparation of 2-alkynyladenosines is illustrated in Scheme 1B. A flame-dried round bottom under nitrogen is charged with 5-(6-Amino-2-iodo-purin-9-yl)-3,4-dihydroxytetrahydrofuran-2-carboxylic acid ethylamide (NECA 2-Iodoadenosine) and a solvent such as DMF. The appropriate alkyne, wherein R is a —(CR¹R²)ₘ Z group, is dissolved in acetonitrile followed by TEA, 5 mole % Pd(PPh₃)₄, and CuI. All solvents are thoroughly degassed.

The solution is allowed to stir for about 24 hours at room temperature, and monitored until complete by HPLC. If the reaction is not complete after this time, additional catalyst, CuI, and TEA are added. After the reaction is complete, the solvents are removed under high-vacuum and the residue taken up in a small amount of DMF. This product is isolated using preparative silica TLC. The product is purified by RP-HPLC.

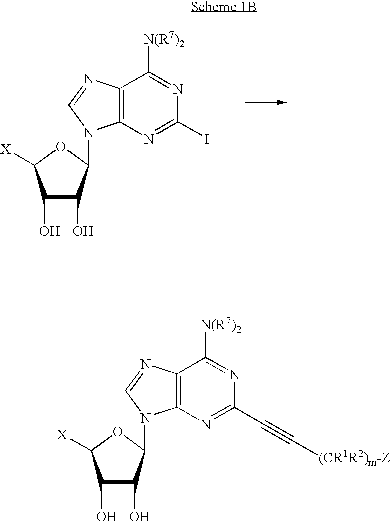

The following abbreviations have been used herein:
2-Aas 2-alkynyladenosines;
¹²⁵I-ABA N⁶-(4-amino-3-¹²⁵iodo-benzyl)adenosine
APCI Atmospheric pressure chemical ionization
ATL146e 4-{3-[6-Amino-9-(5-ethylcarbamoyl-3,4-dihydroxy-tetrahydro-furan-2-yl)-9H-purin-2-yl]-prop-2-ynyl}cyclohexanecarboxylic acid methyl ester;
CCPA 2-chloro-N⁶-cyclopentyladenosine;
CGS21680 2-[4-(2-carboxyethyl)phenethylamino]-5′-N-ethyl-carboxamidoadenosine;
CI-IB-MECA N⁶-3-iodo-2-chlorobenzyladenosine-5′-N-methyl-uronamide;
CPA N⁶-cyclopentyladenosine
DMF dimethylformamide
DMSO dimethylsulfoxide
DMSO-d₆ deuterated dimethylsulfoxide
EtOAc ethyl acetate
eq equivalent
GPCR G protein coupled receptor; hA_{2A} AR, Recombinant human A_{2A} adenosine receptor;
IADO 2-Iodoadenosine
¹²⁵I-APE, 2-[2-(4-amino-3-[¹²⁵I]iodophenyl)ethylamino]-adenosine;
NECA 5′-N-ethylcarboxamidoadenosine;
IB-MECA N⁶-3-iodobenzyladenosine-5′-N-methyluronamide;
2-Iodoadenosine 5-(6-amino-2-iodo-purin-9-yl)-3,4-dihydroxytetra-hydro-furan-2-carboxylic acid ethylamide
HPLC high-performance liquid chromatography
HRMS high-resolution mass spectrometry
¹²⁵I-ZM241385, ¹²⁵I-4-(2-[7-amino-2-[2-furyl][1,2,4]triazolo[2,3-α]-[1,3,5]triazin-5-yl-amino]ethyl)phenol;
INECA 2-iodo-N-ethylcarboxamidoadenosine
LC/MS liquid chromatography/mass spectrometry
m.p. melting point
MHz megahertz
MRS 1220, N-(9-chloro-2-furan-2-yl-[1,2,4]triazolo[1,5-c]-quinazolin-5-yl)-2-phenylacetamide;
MS mass spectrometry
NECA N-ethylcarboxamidoadenosine
NMR nuclear magnetic resonance
RP-HPLC reverse phase high-performance liquid chromatography
TBAF tetrabutylammonium fluoride
TBS tert-butyldimethylsilyl
TBDMSCI tert-butyldimethylsilylchloride
TEA triethylamine
TFA trifluoroacetic acid
THF tetrahydrofuan
TLC thin layer chromatography
p-TSOH para-toluenesulfonic acid
XAC 8-(4-((2-a-minoethyl)aminocarbonyl-methyloxy)-phenyl)-1-3-dipropylxanthine.

The invention will now be illustrated using the following non-limiting examples.

### EXAMPLES

### Materials and Methods

Animals

Male Sprague-Dawley rats weighting 250-300 g were fed a standard laboratory diet and water ad libitum, and kept in cages in a temperature and humidity controlled room with a 12 hour dark-light cycle before and during the experiment. Prior to administration of aspirin, animals were deprived of food for 24 hours but had free access to water. This experimental protocol was approved by the Akita University Animal Care Committee.

Chemicals

4-{3-[6-Amino-9-(5-ethylcarbamoyl-3,4-dihydroxy-tetrahydro-furan-2-yl)-9H-purin-2-yl]-prop-2-ynyl}-cyclohexanecarboxylic acid methyl ester (ATL-146e), was synthesized and purified to >99% purity ATL-146e was dissolved in small volume of dimethylsulfoxide and then diluted>100-fold with physiological saline just before injection.

Effect of ATL-146e on Aspirin-Induced Gastric Mucosal Injury Model

Aspirin-induced gastric injury was produced by intragastric administration of aspirin (200 mg/kg) and HCl (0.15 N, 8 ml/kg). ATL-146e (2.5 or 5 μg/kg, n=5 in each dose) or vehicle was injected intraperitoneally 30 minutes prior to aspirin administration. The animals were sacrificed by stunning and cervical dislocation 3 hours after aspirin administration and the stomach was removed. Gastric mucosal lesions were measured by two independent observers blinded to the treatment. The ulcer index was calculated as the sum of the lengths of all lesions.

Effect of ATL-146e on Myeloperoxidase (MPO) Concentration in the Gastric Mucosa

An assay of gastric mucosal myeloperoxidase concentration was applied to quantify the degree of neutrophil infiltration. Three hundred milligrams of scraped mucosa was homogenized for 30 sec with a polytron homogenizer (PT 1200, KInematica AG, Littau, Switzerland) in 1.0 ml of ice-cold 0.5% hexadecyliltrimethylammonium bromide in 50 mM phosphate buffer (pH 6.0). Hexadecyliltrimethylammonium bromide is used to negate the pseudoperoxidase activity of hemoglobin and to solubilize membrane-bound MPO. The homogenate was sonicated (U50 IKA Werke Grm1H and Co. KG, Staufen, Germany) for 10 seconds, freeze-thawed three times and centrifuged for 20 minutes at 18,000 g. The supernatant was taken for determination of the enzyme activity utilizing an ELISA kit (Bioxytech, Oxis International, Inc. Portland, Oreg., USA). The change in absorbance at 405 nm was measured with a spectrophotometer (Microplate reader model 3550, Bio-Rad, Hercules, Calif., USA). The concentration of myeloperoxidase was expressed as nanogram per mg protein measured using Bradford's method.

Effect of ATL-146e on Gastric Concentration of TNF-α and IL-1β

One hundred milligrams of scraped mucosa was homogenized for 30 sec with a polytron homogenizer (PT 1200, KInematica AG, Littau, Switzerland) in 1.0 ml of ice-cold potassium phosphate buffer (pH 7.4). Following centrifugation at 10,000 g for 10 minutes, aliquots of homogenate supernatants in PBS were obtained by centrifugation (10,000 g for 10 minutes). The total protein was measured by Bradford's method. The concentration of TNF-α and IL-1β in the supernatant of mucosal homogenates was determined by ELISA (R&D systems Inc., Minneapolis, Minn., USA) according to the manufacture's instruction. After color development, optimal density was measured with microplate reader. The concentration of TNF-α and IL-1β were expressed as pictogram/mg protein.

Effect of ATL-146e on Gastric Secretion

Gastric juice was collected using the pylorus-ligation method. Briefly, rats were fasted for 24 hours, placed in restraint cages, and injected intraperitoneal with 5 μg/kg of ATL-146e. Thirty minutes after injection, pylorus ligation was performed. The stomach was removed 3 hours after pylorus ligation, and the gastric contents were collected. Following centrifugation the acid content of the supernatant was determined by titration with 0.01 mol NaOH to pH 7.0 using pH meter (f50 pH Meter; Beckman, Tokyo, Japan), and used to calculate gastric acid secretion in mEq/3 h.

Effect of ATL-146e on Mucosal Content of PGE2

A part of fundic mucosa (about 100 mg) was excised for determination of PGE2 synthesis. The samples were weighed, finely minced with scissors for 15 sec, then suspended in 1.0 ml of 10 mM sodium phosphate buffer (pH 7.4). The samples were then incubated in a shaking bath (37°) for 29 min followed by centrifugation (9000 g for 30 sec). The supernatant was frozen and subsequent determination of PGE2 was performed by radioimmunoassay using PGE2 [125I] RIA kit (Dupont/NEN, Boston, Mass., USA).

Statistical Analysis

All data were expressed as mean ± SEM. Statistical significance was determined by Mann-Whitney U test using Statview-J 4.11 statistical program (Abacus Concepts, Berkeley, Calif., USA) for Macintosh computer. P values of <0.05 were considered statistically significant.

Results

Effect of ATL-146e on Aspirin-Induced Gastric Lesions

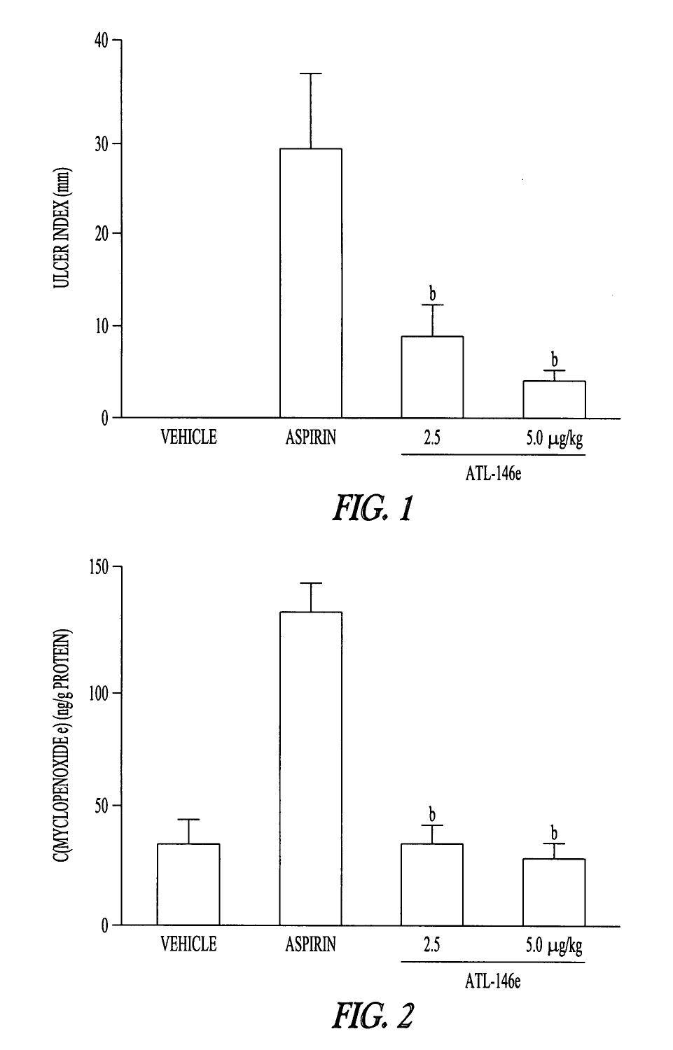

Administration of aspirin resulted in the appearance of linear and dotted erosions in the gastric mucosa of vehicle-treated rats. In contrast, pre-treatment with ATL-146e resulted in smaller erosions. The total length of gastric erosions (ulcer index) in control rats was 29.8±7.7 min. The ulcer index in rats pretreated with ATL-146e was significantly suppressed to 7.0±2.34 mm (2.5 μg/kg, P<0.05), or 3.1±1.42 mm (5.0 μg/kg, P<0.01) (FIG. 1).

Effect of ATL-146e on MPO in Gastric Mucosa

Tissue MPO concentration in the gastric mucosa increased 3 h after the initiation of administration of aspirin. The MPO concentration in normal control animals (2.8±0.2 μg/g protein) increased to 13.8±1.2 μg/g protein in vehicle treated rats. The increment of MPO concentration in the gastric mucosa by aspirin was suppressed by pretreatment with ATL-146e to 2.9±0.35 μg/g protein (2.5 μg/kg, P<0.001) or 2.7±0.14 μg/g protein (5 μg/kg, P<0.001) compared to that in vehicle-treated rats (FIG. 2). The increase in MPO above control levels caused by aspirin was reduced to nearly normal levels after the administration of 2.5 and 5 μg/kg ATL-146e.

Effect of ATL-146e on Gastric of TNF-α and IL-1β

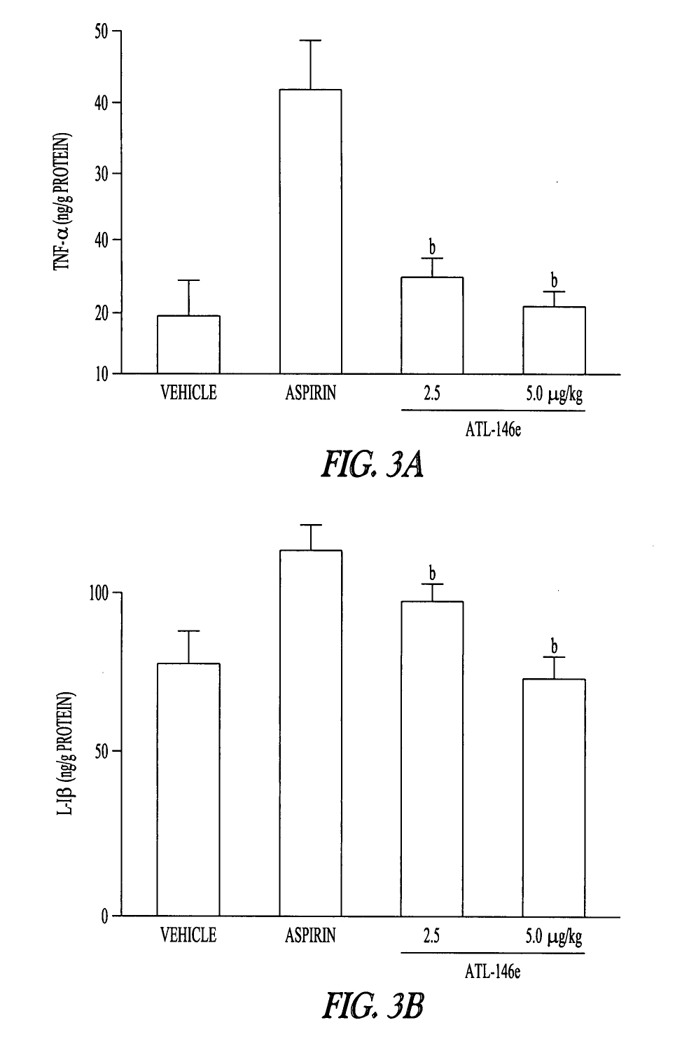

The gastric concentrations of TNF-α and IL-1β were significantly increased 3 h after the administration of aspirin. ATL-146e at the doses of 2.5 and 5.0 μg/kg significantly suppressed the increment of tissue TNF-α and IL-1β in the gastric mucosa by the administration of aspirin (FIG. 3). The increase in TNF-α above the control levels caused by aspirin was reduced by the administration of 2.5 and 5 μg/kg ATL-146e by 90.3% and 99.7%, respectively. The increase in IL1-β above the control levels caused by aspirin was reduced by the administration of 2.5 and 5 μg/kg ATL-146e by 64.0% and 97.2%, respectively.

Effect of ATL-146e on Gastric Secretion

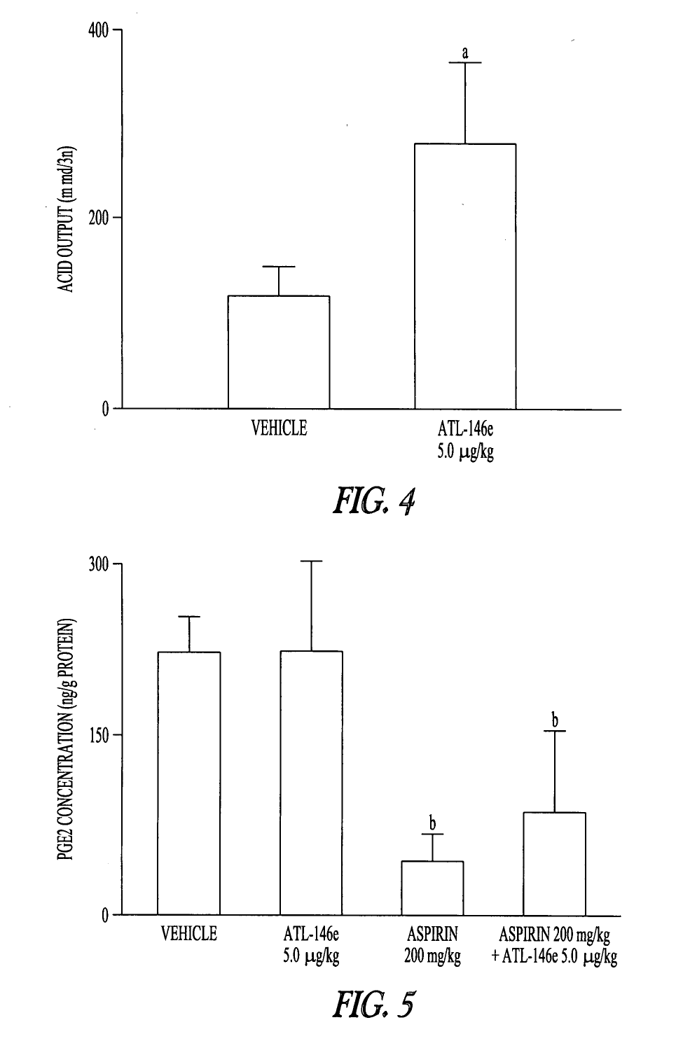

ATL-146e caused a threefold increase in gastric acid output from 114 to 326 mmolq/3 h (P<0.05, FIG. 4).

Effect of ATL-146e on Mucosal of PGE2

The concentration of PGE2 was 255.3±64.7 ng/g in vehicle-treated rats (group A) and 47.4±5.6 ng/g in aspirin treated animals (group C) (82.3% reduction, P<0.05). ATL-146e (5 μg/kg) administration did not interfere with the reduction of gastric PGE2 concentration induced by aspirin (P>0.05 vs. aspirin alone) (FIG. 5).

Discussion

The results of the study presented herein study clearly showed that a single bolus injection of the adenosine A_{2A} agonist, ATL-146e, 30 min prior to the administration of aspirin, could effectively reduce the extent of gastric mucosal lesions. This protection is correlated with the inhibition of neutrophil infiltration into the gastric mucosal tissue and production of pro-inflammatory cytokines in gastric mucosal tissue. Previous studies have shown that gastric mucosal MPO concentration, a biochemical indicator of neutrophils, increases with the development of gastric mucosal lesions. These findings suggest that most of the stimuli for aspirin-induced gastric injury that is susceptible to the inhibition by ATL-146e may occur early after the administration of aspirin.

Recent studies reported that gastric mucosal lesions can be reduced by the administration of antibodies against TNF-α. Therefore, it is possible that infiltration of neutrophils in rat gastric mucosa after the administration of aspirin could occur in response to the gastric production of pro-inflammatory cytokines, resulting in the development of gastric mucosal lesions secondary to neutrophil accumulation. Anti-inflammatory and tissue protective effects of ATL-146e in the models of ischemia-reperfusion injury have been reported.

Adenosine has been identified as an endogenous anti-inflammatory agent because the activation of the A_{2A} receptor is known to increase intracellular cAMP levels and to reduce diverse leukocyte functions. Ross et al. (1999) demonstrated that ATL-146e protects lung from reperfusion injury by reducing neutrophil sequestration. A recent study reported that ATL-146e inhibits water immersion stress-induced gastric injury due to the inhibition of neutrophil accumulation and reduction of pro-inflammatory cytokine production. These findings led us to examine the effect of ATL-146e on aspirin induced gastric mucosal lesion. In the study presented herein, it is demonstrated that MPO concentration, an index of tissue-associated neutrophil accumulation, increased in the gastric mucosa 3 h after the administration of aspirin. The increased MPO concentration was significantly inhibited by treatment with ATL-146e.

TNF-α is a pro-inflammatory cytokine and has recently been shown to be a mediator of NSAIDs-induced gastric mucosal injury. Also, TNF-α is a cytokine that stimulates neutrophil adherence by inducing synthesis and expression of adhesion molecules on endothelial cells and neutrophils. TNF-α augments neutrophil-derived superoxide generation and upregulates the expression of adhesion molecules on neutrophil and endothelium, and stimulates production of IL-1β, leading to neutrophil accumulation. Furthermore, studies on experimental models have shown that intravenous administration of TNF-α produces extensive neutrophil infiltration within the microvasculature of the digestive tract. In addition, portal infusion of TNF-α causes gastric and small intestinal damage in rats. The study presented herein demonstrated that ATL-146e treatment could inhibit increase of TNF-α and IL-1β concentration in the gastric mucosa after the administration of aspirin. These findings strongly support that ATL-146e attenuates aspirin-induced neutrophil accumulation by inhibiting production of pro-inflammatory cytokines.

Okusa et al. (2000) reported that ATL-146e inhibits ischemic reperfusion injury of kidney not only by reducing neutrophil accumulation, but also by reducing the expression of the adhesion molecules, P-selectin, and ICAM-1 on the reperfused vascular endothelium. Andrews et al. (1994) also reported that the expression of ICAM-1 on endothelial cells is increased by NSAIDs. Therefore, the anti-ulcer effect of ATL-146e may occur via inhibition of neutrophil adhesion.

In addition, various antisecretory agents, such as H2-receptor antagonists and proton pump inhibitors prevent gastric lesions. However, as shown herein, ATL-146e could significantly increase gastric acid secretion. These data indicate that the protective effect of ATL-146e is not due to reduced gastric acid secretion. Also, it has been reported that PGE2 prevents gastric mucosal damage by aspirin in human beings and animals. However, the protective effect of ATL-146e is not dependent on gastric mucosal prostaglandin synthesis, since pretreatment with ATL-146e, which reduces gastric damage, has no effect on the gastric mucosal prostaglandin concentration. Therefore, inhibition of mucosal lesions by ATL-146e cannot be attributed to gastric acid inhibition and/or prostaglandin synthesis.

In conclusion, the potent and selective adenosine A_{2A} receptor agonist, ATL-146e, significantly inhibits gastric mucosal injury induced by aspirin in rats. This effect may be due in part to a reduction in neutrophil infiltration into the gastric mucosa and inhibition of proinflammatory cytokines production. Thus, modulation of adenosine A_{2A} receptor activity by specific agonists may be clinically useful for the therapy of NSAIDs-induced gastric mucosal damage.

Citations

Hall J, et al. Arthritis Rheum 1994; 37: 1132-1137.
Rashad S, et al. Lancet 1989; 2: 519-522.
Beck P L, et al. Gastroenterology 2000; 119: 699-705.
Yoshikawa T, et al. Gut 1993; 34: 732-737.
Takeuchi K, et al. Digestion 1991; 49: 175-184.
Andrews F J, et al. Am J Physiol 1994; 266: G657-664.
Wallace J L, et al. Am J Physiol 1990; 259: G462-G467.
Yoshida N, et al. Dig Dis Sci 1995; 40: 2300-2304.
Olah M E, et al. Annu Rev Pharmacol Toxicol 1995; 35: 581-606.
Linden J. et al. Annu Rev Pharmacol Toxicol 2001; 41: 775-787.
Sullivan G W and Linden J. Drug Develop Res 1998; 45: 103-112.
Cronstein B N. J Appl Physiol 1994; 76: 5-13.
Okusa M D, et al. Am J Physiol Renal Physiol 2000; 279: F809-818.
Ross S D, et al. J Heart Lung Transplant 1999; 18: 994-1002.
Jordan J E, et al. J Pharmacol Exp Ther 1997; 280: 301-309.
Harada N, et al. J Pharmacol Exp Ther 2000; 294: 1034-1042.
Link A A, et al. J Immunol 2000; 164: 436-442.
Hasko G, et al. FASEB J 2000; 14: 2065-2074.
Odashima M, et al. J Gastroenterol Hepatol 2005; 20: 275-280.
Murphree L J, et al. Mol Pharmacol 2002; 61: 455-462.
Rieger J M, et al. J Med Chem 2001; 44: 531-539.
Brodie D A and Hanson H M. Gastroenterology 1960; 38: 353-360.
Bradford M M. Anal Biochem 1976; 72: 248-254.
Shay H, et al. Gastroenterology 1945; 22: 1077-1061.
Krawisz J E, et al. Gastroenterology 1984; 87: 1344-1350.
Hamaguchi M, et al. Dig Dis Sci 2001; 46: 2708-2715.
Okusa M D, et al. Kidney Int 2001; 59: 2114-2125.
Day Y J, et al. Am J Physiol Gastrointest Liver Physiol 2004; 286: G285-93.
Day Y J, et al. J Clin Invest 2003; 112: 883-891.
Sullivan G W, et al. J Immunol 1990; 145: 1537-1544.
Santucci L, et al. Gut 1994; 35: 909-915.
Yoshikawa T, et al. Int J Immunopharmacol 1992; 14: 1391-1398.
Kokura S, et al. Circ Res 2000; 86: 205-213.
Kahky M P, et al. J Surg Res 1990; 49: 138-145.
Hamajima E, et al. Dig Dis Sci 1994; 39: 713-720.
Appleyard C B, et al. Am J Physiol 1996; 270: G42-G48.

All cited publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A therapeutic method for treating gastric lesions, comprising administration to a patient in need thereof an effective amount of an A_{2A} adenosine receptor agonist.

2. The method of claim 1, wherein the gastric lesion is caused by stress, bacterial infection, smoking, alcohol, or a non-steroidal anti-inflammatory drug.

3. The method of claim 1, wherein the gastric lesion is caused by contact with a non-steroidal anti-inflammatory drug.

4. The method of claim 1, wherein the non-steroidal anti-inflammatory drug is aspirin.

5. The method of claim 1, wherein the A_{2A} adenosine receptor agonist is a compound having formula (I): 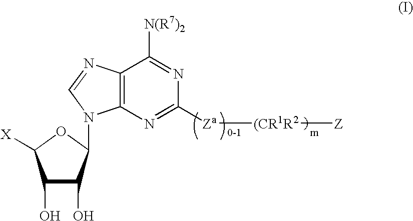
wherein
Z^{a} is C≡C, O, NH, or NHN═CR^{3a};
Z is CR³R⁴R⁵ or NR⁴R⁵;
each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(S)N(R^{b})—, R^{a}OC(═S)—, R^{a}C(═S)—, —SSR^{a}, R^{a}S(═O)—, R^{a}S(═O)₂—, or —N═NR^{b};
each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-; or
R¹ and R² and the atom to which they are attached is C═O, C═S or C═NR^{d};
R⁴ and R⁵ are independently H or (C₁-C₈)alkyl; or
R⁴ and R⁵ together with the atom to which they are attached form a saturated, partially unsaturated, or aromatic ring that is mono-, bi-, or polycyclic having 3, 4, 5, 6, 7, 8, 9 or 10 ring atoms optionally having 1, 2, 3, or 4 heteroatoms selected from non-peroxide oxy (—O—), thio (—S—), sulfinyl (—SO—), sulfonyl (—S(O)₂—) or amine (—NR^{b}—) in the ring;
wherein R⁴ and R⁵ are independently substituted with 0-3 R⁶ groups or any ring comprising R⁴ and R⁵ is substituted with from 0 to 14 R⁶ groups; wherein each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle (C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring comprising from 1-6 atoms and optionally comprising 1, 2, 3, or 4 heteroatoms selected from non-peroxide oxy (—O—), thio (—S—), sulfinyl (—SO—), sulfonyl (—S(O)₂—) or amine (—NR^{b}—) in the ring;
R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, R^{a}OC(═S)—, R^{a}C(═S)—, —SSR^{a}, R^{a}S(═O)—, R^{a}S(═O)₂—, or —NNR^{b}; or if the ring formed from CR⁴R⁵ is aryl or heteroaryl or partially unsaturated then R³ can be absent;
R^{3a} is hydrogen, (C₁-C₈)alkyl or aryl;
each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;
X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c}); or
X is an aromatic ring of the formula: 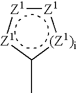
each Z¹ is non-peroxide oxy (—O—), S(O)₀₋₂, —C(R⁸)—, or amine (—NR⁸—), provided that at least one Z¹ is non-peroxide oxy (—O—), thio (—S—), sulfinyl (—SO—), sulfonyl (—S(O)₂—) or amine (—NR⁸—);
each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;
wherein any of the alkyl, cycloalkyl, heterocycle, aryl, or heteroaryl, groups of R¹, R², R³, R^{3a}, R⁶, R⁷ and R⁸ is optionally substituted on carbon with one or more substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, R^{a}OC(═S)—, R^{a}C(═S)—, —SSR^{a}, R^{a}S(═O)ₚ—, R^{b}R^{c}NS(O)_{P}—, and —N═NR^{b};
wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;
each R^{a}, R^{b}, and R^{c}C is independently hydrogen, (C₁-C₁₂)alkyl, (C₁-C₈)alkoxy-(C₁-C₈)alkyl-, (C₃-C₈)cycloalkyl, (C₁-C₈)alkylthio-(C₁-C₈)alkyl-, amino acid, aryl, aryl(C₁-C₈)alkylene, heteroaryl, or heteroaryl(C₁-C₈)alkylene;
alternatively R^{b} and R^{c}, together with the nitrogen to which they are attached, form a pyrrolidino, piperidino, morpholino, or thiomorpholino ring;
R^{d} is hydrogen or (C₁-C₆)alkyl;
i is 1 or 2
m is 0 to 8 and p is 0 to 2;
provided that m is at least 1 when Z is NR⁴R⁵; or
a pharmaceutically acceptable salt thereof.

6. The method of claim 5, wherein the compound is of formula (Ia): 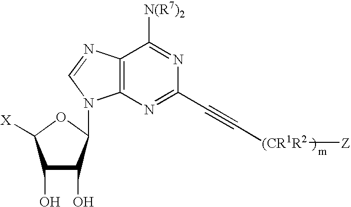
R¹ is hydrogen, —OH, —CH₂OH, —OMe, —OAc, —NH₂, —NHMe, —NMe₂ or —NHAc;
R² is hydrogen, (C₁-C₈)alkyl, cyclopropyl, cyclohexyl or benzyl;
R³ is hydrogen, OH, OMe, OAc, NH₂, NHMe, NMe₂ or NHAc;
CR⁴R⁵ or NR⁴R⁵ is optionally substituted with 0-2 R⁶ and is cyclopentane, cyclohexane, piperidine, dihydro-pyridine, tetrahydro-pyridine, pyridine, piperazine, tetrahydro-pyrazine, dihydro-pyrazine, pyrazine, dihydro-pyrimidine, tetrahydro-pyrimidine, hexahydro-pyrimidine, pyrazine, imidazole, dihydro-imidazole, imidazolidine, pyrazole, dihydro-pyrazole, or pyrazolidine; or
the ring CR⁴R⁵ or NR⁴R⁵ is optionally substituted with 0-2 R⁶ and is selected from the group consisting of: 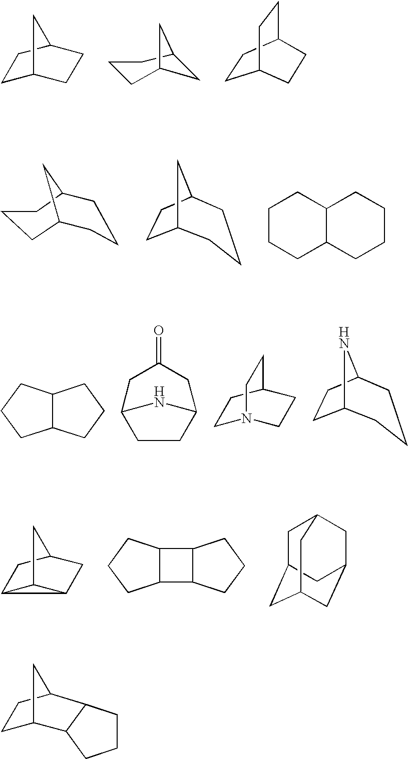
R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;
R^{a} and R^{b} are independently hydrogen, (C₃-C₄)-cycloalkyl, (C₁-C₈)alkyl, aryl or aryl(C₁-C₈)alkylene;
each R⁷ is independently hydrogen, (C₁-C₈)alkyl, aryl, aryl(C₁-C₈)alkylene, or heteroaryl(C₁-C₈)alkylene;
R⁸ is methyl, ethyl, propyl, 2-propenyl, cyclopropyl, cyclobutyl, cyclopropylmethyl, —(CH₂)₂CO₂CH₃, or —(CH₂)₂₋₃OH;
X is —CH₂OR^{a}, —CO₂R^{a}, —CH₂OC(O)R^{a}, or —C(O)NR^{b}R^{c}; or
X is selected from: 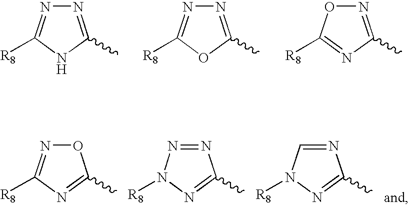
m is 0, 1, or 2;
or a pharmaceutically acceptable salt thereof.

7. The method of claim 6, wherein
R¹ is hydrogen, OH, OMe, or NH₂;
R² is hydrogen, methyl, ethyl or propyl;
R³ is hydrogen, OH, OMe, or NH₂;
the ring CR⁴R⁵ or NR⁴R⁵ is selected from the group consisting of: 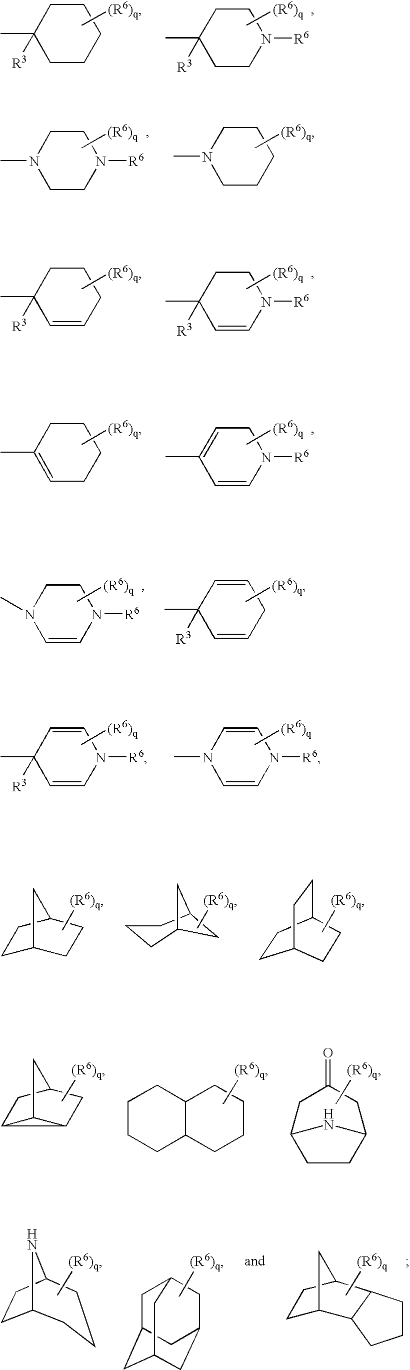
q is from 0 to 4;
R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;
R^{a} and R^{b} are independently hydrogen, methyl, ethyl, propyl, butyl, ethylhexyl, cyclopropyl, cyclobutyl, phenyl or benzyl;
N(R⁷)₂ is amino, methylamino, dimethylamino; ethylamino; pentylamino, diphenylethylamino, (pyridinylmethyl)amino, (pyridinyl)(methyl)amino, diethylamino or benzylamino; and, R⁸ is methyl, ethyl, propyl, or cyclopropyl;
X is —CH₂OR^{a} or —C(O)NR^{b}R^{c}; or
X is selected from: 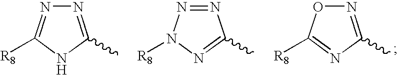
or a pharmaceutically acceptable salt thereof.

8. The method of claim 7, wherein:
R¹ is hydrogen, OH, or NH₂;
R² is hydrogen or methyl;
R³ is hydrogen, OH, or NH₂;
the ring CR⁴R⁵ or NR⁴R⁵ is selected from the group consisting of: 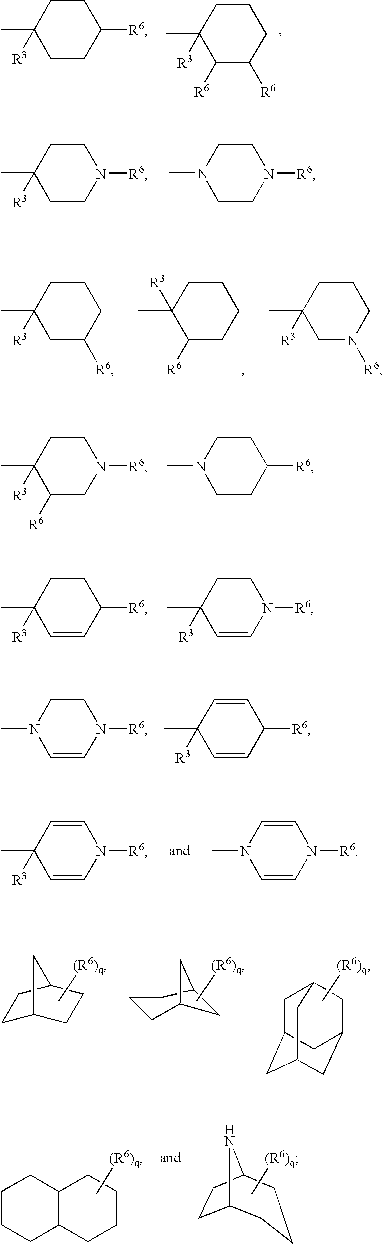
where q is from 0 to 2;
R⁶ is hydrogen, methyl, ethyl, t-butyl, , phenyl, —CO₂R^{a}—CONR^{b}R^{c}, or R^{a}C(═O)—;
R^{b} is H;
R^{a} is methyl, ethyl, propyl, butyl, pentyl, ethylhexyl cyclopropyl, or cyclobutyl;
—N(R⁷)₂ is amino, methylamino, dimethylamino; ethylamino; diethylamino or benzylamino;
or a pharmaceutically acceptable salt thereof.

9. The method of claim 8, wherein:
R¹ is hydrogen or OH;
R² is hydrogen;
R³ is hydrogen or OH;
the ring CR⁴R⁵ or NR⁴R⁵ is selected from the group consisting of: 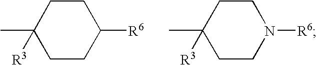
R⁶ is hydrogen, methyl, ethyl, —CO₂R^{a}, or —CONR^{b}R^{c};
R^{b} is H;
R^{a} is methyl, ethyl, i-propyl, i-butyl, tert-butyl, or cyclopropyl;
N(R⁷)₂ is amino, or methylamino;
X is —CH₂OH, 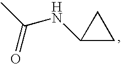
C(O)NHCH₃, or —C(O)NHCH₂CH₃;
or a pharmaceutically acceptable salt thereof.

10. The method of claim 5, wherein the ring comprising R⁴ and R⁵ is 2-methyl cyclohexane, 2,2-dimethylcyclohexane, 2-phenyl cyclohexane, 2-ethylcyclohexane, 2,2-diethylcyclohexane, 2-tert-butyl cyclohexane, 3-methyl cyclohexane, 3,3-dimethylcyclohexane, 4-methyl cyclohexane, 4-ethylcyclohexane, 4-phenyl cyclohexane, 4-tert-butyl cyclohexane, 4-carboxymethyl cyclohexane, 4-carboxyethyl cyclohexane, 3,3,5,5-tetramethyl cyclohexane, 2,4-dimethyl cyclopentane, 4-cyclohexanecarboxylic acid, 4-cyclohexanecarboxylic acid esters, 4-methyloxyalkanoyl-cyclohexane, 4-piperidine-1-carboxylic acid methyl ester, 4-piperidine-1-carboxylic acid tert-butyl ester, 4-piperidine, 4-piperazine-1-carboxylic acid methyl ester, 4-piperidine-1-carboxylic acid tert-butylester, 1-piperidine-4-carboxylic acid methyl ester, 1-piperidine-4-carboxylic acid tert-butyl ester, tert-butylester, 1-piperidine-4-carboxylic acid methyl ester, or 1-piperidine-4-carboxylic acid tert-butyl ester, 3-piperidine-1-carboxylic acid methyl ester, 3-piperidine-1-carboxylic acid tert-butyl ester, 3-piperidine, 3-piperazine-1-carboxylic acid methyl ester, 3-piperidine-1-carboxylic acid tert-butylester, 1-piperidine-3-carboxylic acid methyl ester, or 1-piperidine-3-carboxylic acid tert-butyl ester;
or a pharmaceutically acceptable salt thereof.

11. The method of claim 5, wherein the A_{2A} adenosine receptor agonist is 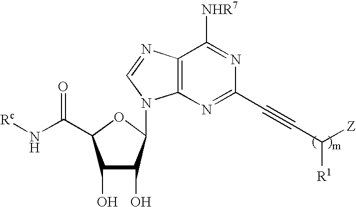
wherein R^{c} is Et; R₇ is H and —(R¹)ₘ-Z is selected from the group consisting of: 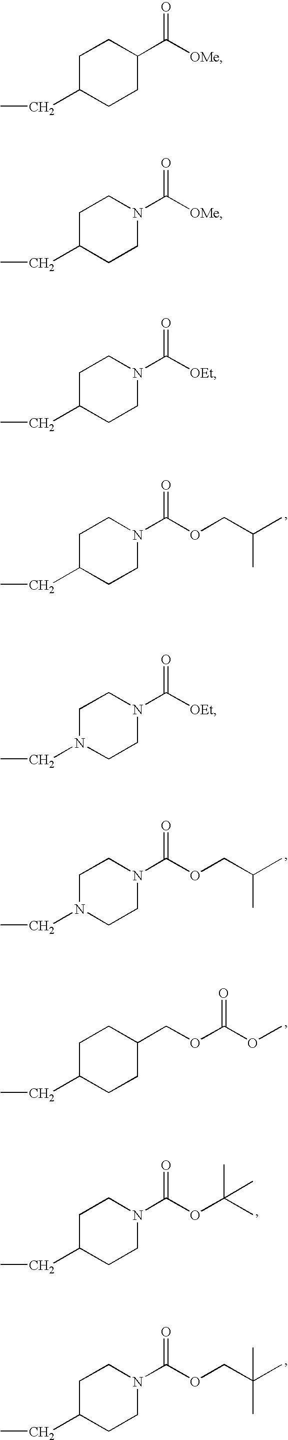 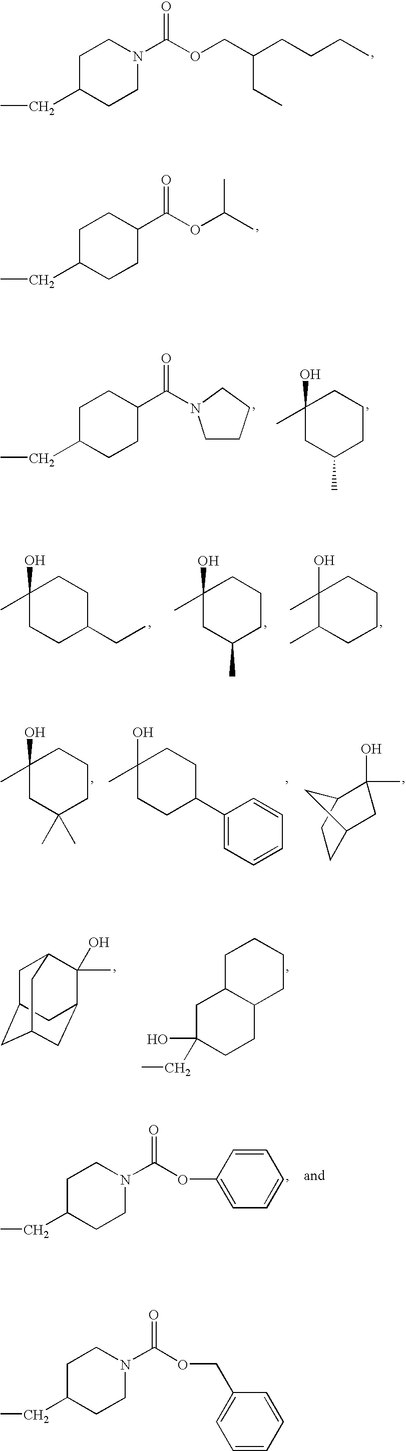
or a pharmaceutically acceptable salt thereof.

12. The method of claim 5, wherein the A_{2A} adenosine receptor agonist is 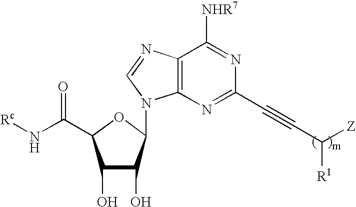
wherein R^{c} is cPr; R₇ is H and —(R¹)ₘ-Z is selected from the group consisting of: 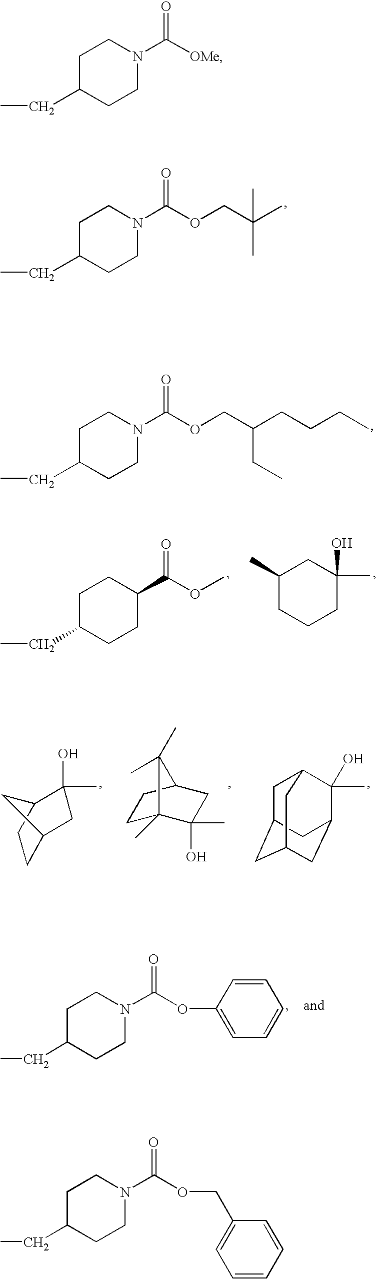
or a pharmaceutically acceptable salt thereof.

13. The method of claim 11, wherein R^{c} is Et, R⁷ is H and —(R¹)ₘ-Z is

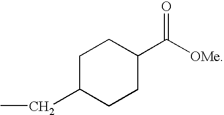

14. The method of claim 5, wherein the A_{2A} adenosine receptor agonist is formula (Ib)-(Id) or a pharmaceutically acceptable salt thereof:

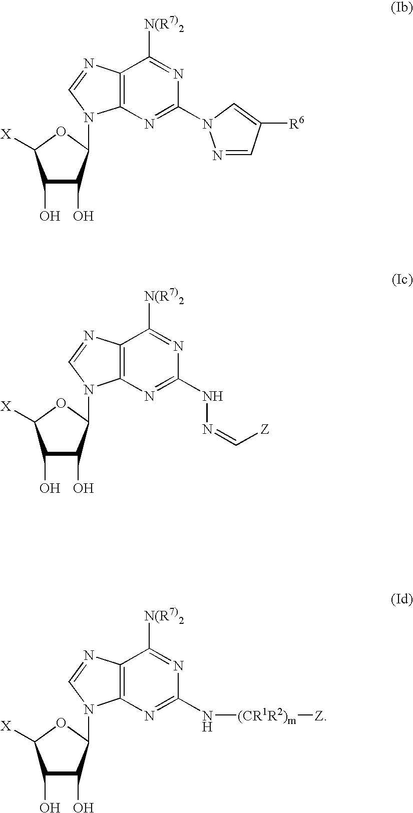

15. The method of claim 14, wherein the A_{2A} adenosine receptor agonist is selected from: 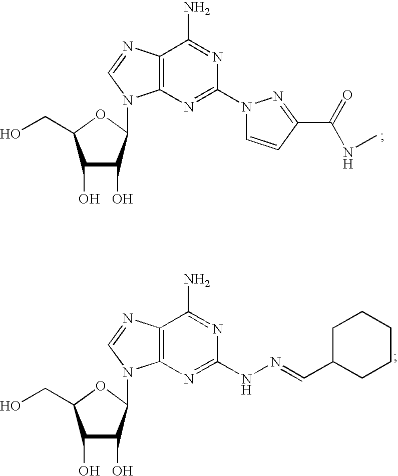
or a pharmaceutically acceptable salt thereof.

16. The method of claim 1, wherein the A_{2A} adenosine receptor agonist is a compound of the following formula or a pharmaceutically acceptable salt thereof:

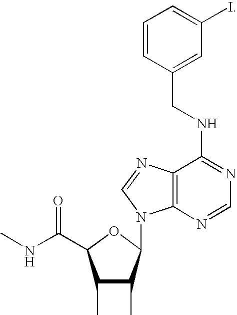

17. The method of claim 1, wherein the A_{2A} adenosine receptor agonist is administered orally, intravenously, intraperioteneally, or transdermally.
